(19) United States
(12) Patent Application Publication
(10) Pub. No.: US 2008/0076778 A1
(43) Pub. Date: Mar. 27, 2008

(54) METHODS FOR DESIGNING PARP INHIBITORS AND USES THEREOF

(75) Inventors: Valeria Ossovskaya, San Francisco, CA (US); John Burnier, Pacifica, CA (US); Barry Sherman, Hillsborough, CA (US); Max Totrov, San Diego, CA (US)

Correspondence Address: WILSON SONSINI GOODRICH & ROSATI, 650 PAGE MILL ROAD, PALO ALTO, CA, 94304-1050, US

(73) Assignee: BiPar Sciences, Inc., Brisbane, CA (US)

(21) Appl. No.: 11/850,612

(22) Filed: Sep. 5, 2007

Related U.S. Application Data
(60) Provisional application No. 60/842,470, filed on Sep. 5, 2006.

(51) Int. Cl. A61K 31/37; A61K 31/4025; A61K 31/4178; A61K 31/4433; A61K 31/496; A61P 11/00; A61P 15/00; A61P 25/00; A61P 29/00; A61P 3/00; A61P 35/00; A61P 5/00; C07D 311/02; C07D 405/10; G06G 7/48

(52) U.S. Cl. 514/254.11; 514/337; 514/389; 514/422; 514/457; 544/376; 546/283.1; 548/311.4; 548/525; 549/283; 549/288; 703/11

(57) ABSTRACT

The present invention relates to a computer-assisted method of a designing of a PARP inhibitor comprising: a) determining an interaction between a candidate PARP protein and a known PARP inhibitor by evaluating a binding of the PARP protein to the known PARP inhibitor; b) based on the interaction, designing a candidate PARP inhibitor; c) determining an interaction between the PARP protein and the candidate PARP inhibitor by evaluating a binding of the PARP protein to the candidate PARP inhibitor; and d) concluding that the candidate PARP inhibitor inhibits the PARP protein wherein the conclusion is based on the interaction of step c). The invention also provides methods for treatment of diseases with the candidate PARP inhibitors.

### CROSS-REFERENCE

This application claims the benefit of U.S. Provisional Application No. 60/842,470, filed Sep. 5, 2006, which is incorporated herein by reference in its entirety.

### BACKGROUND OF THE INVENTION

PARP (poly-ADP ribose polymerase) participates in a variety of DNA-related functions including cell proliferation, differentiation, apoptosis, DNA repair and also has effects on telomere length and chromosome stability (d'Adda di Fagagna et al, 1999, Nature Gen., 23(1): 76-80). Oxidative stress-induced over activation of PARP consumes NAD+ and consequently ATP, culminating in cell dysfunction or necrosis. This cellular suicide mechanism has been implicated in the pathomechanism of cancer, stroke, myocardial ischemia, diabetes, diabetes-associated cardiovascular dysfunction, shock, traumatic central nervous system injury, arthritis, colitis, allergic encephalomyelitis, and various other forms of inflammation. PARP has also been shown to associate with and regulate the function of several transcription factors. The multiple functions of PARP make it a target for a variety of serious conditions including various types of cancer and neurodegenerative diseases.

PARP-inhibition therapy represents an effective approach to treat a variety of diseases. In cancer patients, PARP inhibition may increase the therapeutic benefits of radiation and chemotherapy. Targeting PARP inhibition may prevent tumor cells from repairing DNA themselves and developing drug resistance, which may render the cells more sensitive to existing cancer therapies. PARP inhibitors have demonstrated the ability to increase the effect of various chemotherapeutic agents (e.g. methylating agents, DNA topoisomerase inhibitors, cisplatin etc.), as well as radiation, against a broad spectrum of tumors (e.g. glioma, melanoma, lymphoma, colorectal cancer, head and neck tumors).

The identification of PARP inhibitors can be accomplished by using methods such as the screening of large numbers of random libraries of natural and/or synthetic compounds. However, this method is inefficient in that it typically results in a small number of “hits” and it is constrained by the limitations imposed in screening large numbers of compounds in laboratory assays. Another method of such identification is structure-based drug design (“SBDD”). SBDD comprises a number of integrated components, including, structural information (e.g., spectroscopic data such as X-ray or magnetic resonance information, relating to enzyme structure and/or conformation, enzyme-ligand interactions, etc.), computer modeling, medicinal chemistry, and biological testing (both in vitro and in vivo). These components, each alone or in combination, are useful for accelerating the drug discovery process, for gaining insight into disease and disease processes, and for providing a more efficient method for identifying drug candidates.

Accordingly, the present invention provides compositions and methods related to design of PARP inhibitors and methods of treatment thereof.

### SUMMARY OF THE INVENTION

One aspect of the invention relates to a computer-assisted method of designing a PARP inhibitor comprising: a) determining an interaction between a candidate PARP protein and a known PARP inhibitor by evaluating a binding of the PARP protein to the known PARP inhibitor; b) based on the interaction, designing a candidate PARP inhibitor; c) determining an interaction between the PARP protein and the candidate PARP inhibitor by evaluating a binding of the PARP protein to the candidate PARP inhibitor; and d) concluding that the candidate PARP inhibitor inhibits the PARP protein wherein the conclusion is based on the interaction of step c). In some preferred embodiments, the PARP protein is PARP 1 protein. In some embodiments, the PARP protein is a three-dimensional structure derived from a crystal of the PARP protein and wherein the three dimensional structure comprises a binding domain of the PARP protein. In some embodiments, the binding domain of the PARP protein is selected from the group consisting of DNA binding domain, automodification domain, and catalytic domain. In some preferred embodiments, the binding domain of the PARP protein is a catalytic domain.

In some preferred embodiments of the aforementioned aspect of the present invention, the designing is performed in conjunction with computer modeling. In some embodiments, the designing involves replacing a substituent on the known PARP inhibitor with another substituent wherein the other substituent improves the binding of the candidate PARP inhibitor with the PARP protein. In some embodiments, the interaction is steric interaction, van der Waals interaction, electrostatic interaction, solvation interaction, charge interaction, covalent bonding interaction, non-covalent bonding interaction, entropically favorable interaction, enthalpically favorable interaction, or a combination thereof. In some embodiments, the candidate PARP inhibitor is an analog of a known PARP inhibitor. In some embodiments, the candidate PARP inhibitor contains a hydrophilic group. In some embodiments, the hydrophilic group contains at least one nitrogen. In some preferred embodiments, the known PARP inhibitor is an iodonitrocoumarin. Preferably, the iodonitrocoumarin is 5-iodo-6-nitrocoumarin. In some preferred embodiments, the candidate PARP inhibitor is an analog of the iodonitrocoumarin.

Some preferred embodiments of the aforementioned aspect of the present invention further comprise a step of chemically synthesizing the candidate PARP inhibitor. In some embodiments, the step further comprises evaluating a PARP inhibiting activity of the candidate PARP inhibitor and selecting the candidate PARP inhibitor based on the evaluation. In some embodiments, the evaluating the PARP inhibiting activity involves an assay technique.

In some embodiments, the candidate PARP inhibitor is a compound of formula I, its pharmaceutically acceptable salts or prodrugs thereof: 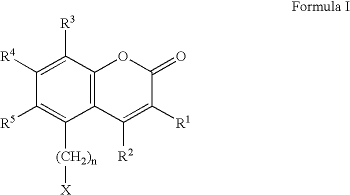
wherein n=0-10; R¹, R², R³, R⁴, R⁵ and X are independently selected from the group consisting of hydrogen, hydroxy, optionally substituted amine, carboxyl, ester, nitroso, nitro, halogen, optionally substituted (C₁-C₆) alkyl, optionally substituted (C₁-C₆) alkoxy, optionally substituted (C₃-C₇) cycloalkyl, optionally substituted (C₃-C₇) heterocyclic, and optionally substituted aryl; and wherein at least two of the R¹, R², R³, R⁴, and R⁵ substituents are always hydrogen. In some embodiments, n is 0, 1, 2, 3, 4, 5, 6, 7, 8, 9 or 10. In some embodiments, n is 1, 2, 3, 4, 5, 6, 7, 8, 9 or 10. In some embodiments, the halogen is selected from the group consisting of I, Br and Cl. In some embodiments, the halogen is Cl or Br. In some embodiments wherein R⁵ is amino, nitro or nitroso, n is 1, 2, 3, 4, 5, 6, 7, 8, 9 or 10. In some embodiments in which R⁵ is amino, nitro or nitroso, and n is 0, 1, 2, 3, 4, 5, 6, 7, 8, 9 or 10, X is optionally substituted (C₁-C₆) alkyl, optionally substituted (C₁-C₆) alkoxy, optionally substituted (C₃-C₇) cycloalkyl, optionally substituted (C₃-C₇) heterocyclic or optionally substituted aryl.

In some embodiments, the candidate PARP inhibitor is a compound of formula II or its pharmaceutically acceptable salts or prodrugs:

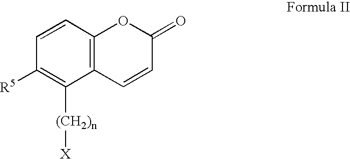

wherein R⁵ is selected from the group consisting of carboxyl, nitroso, and nitro; and X is selected from the group consisting of optionally substituted (C₁-C₇) alkyl, optionally substituted (C₁-C₆) alkoxy, optionally substituted (C₃-C₇) cycloalkyl, optionally substituted (C₃-C₇) heterocyclic, and optionally substituted aryl.

In some embodiments, the candidate PARP inhibitor is a compound of formula III or its pharmaceutically acceptable salts or prodrugs:

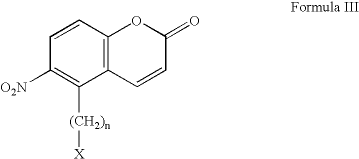

wherein n=0-10, and wherein X is selected from the group consisting of optionally substituted (C₃-C₇) cycloalkyl, optionally substituted (C₃-C₇) heterocyclic, and optionally substituted aryl.

In some embodiments, the optionally substituted aryl is substituted with an optionally substituted alkyl. In some embodiments, optionally substituted alkyl is substituted with a substituent selected from the group consisting of alkylamine, pyrrole, dihydropyrrole, or pyrrolidene.

In some preferred embodiments, the candidate PARP inhibitor is a compound of formula IIIa or its pharmaceutically acceptable salts or prodrugs:

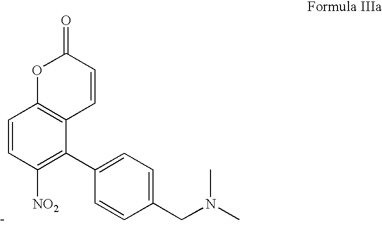

In some preferred embodiments, the candidate PARP inhibitor is a compound of formula IIIb or its pharmaceutically acceptable salts or prodrugs:

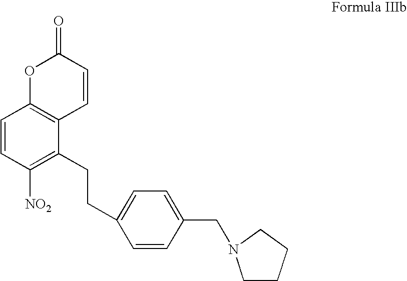

In some preferred embodiments, the optionally substituted (C₃-C₇) heterocyclic is a five membered heterocyclic ring or a six membered heterocyclic ring. In some embodiments, the optionally substituted (C₃-C₇) heterocyclic contains at least one nitrogen. In some embodiments, the optionally substituted (C₃-C₇) heterocyclic is selected from the group consisting of azeridine, azetidine, pyrrole, dihydropyrrole, pyrrolidene, pyrazole, pyrazoline, pyrazolidine, imidazole, benzimidazole, triazole, tetrazole, oxazole, isoxazole, benzoxazole, oxadiazole, oxazoline, oxazolidine, thiazole, isothiazole, pyridine, dihydropyridine, tetrahydropyridine, quinazoline, pyrazine, pyrimidine, pyridazine, quinoline, isoquinoline, triazine, tetrazine, and piperazine.

In some embodiments, the optionally substituted (C₃-C₇) heterocyclic is substituted with a substituent selected from the group consisting of optionally substituted (C₁-C₆) alkyl, optionally substituted (C₁-C₆) alkoxy, optionally substituted (C₃-C₇) cycloalkyl, optionally substituted (C₃-C₇) heterocyclic, and optionally substituted aryl.

In some preferred embodiments, the candidate PARP inhibitor is a compound of formula IIIc or its pharmaceutically acceptable salts or prodrugs:

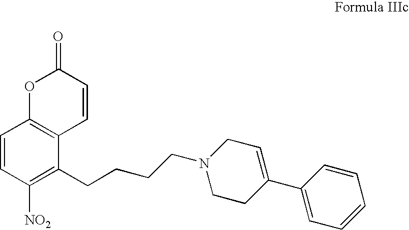

In some preferred embodiments, the candidate PARP inhibitor is a compound of formula IIId or its pharmaceutically acceptable salts or prodrugs:

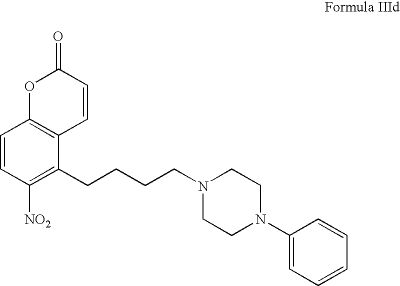

In some preferred embodiments, the candidate PARP inhibitor is a compound of formula IIIe or its pharmaceutically acceptable salts or prodrugs:

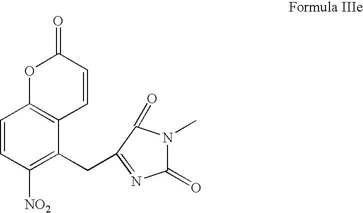

In some preferred embodiments, the candidate PARP inhibitor is a compound of formula IIIf or its pharmaceutically acceptable salts or prodrugs:

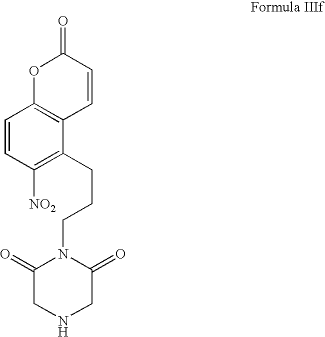

Another aspect of the present invention relates to a computer system containing a set of information to perform a design of a PARP inhibitor having a user interface comprising a display unit, the set of information comprising: a) logic for inputting an information regarding a binding of a PARP protein to a known PARP inhibitor; b) logic for designing a candidate PARP inhibitor based on the binding of the PARP protein and known PARP inhibitor; c) logic for determining an information regarding a binding of the PARP protein to the candidate PARP inhibitor; and d) logic for making a conclusion regarding the PARP inhibitory properties of the candidate PARP inhibitor based on the determination of step c).

Another aspect of the present invention relates to a computer-readable storage medium containing a set of information for a general purpose computer having a user interface comprising a display unit, the set of information comprising: a) logic for inputting an information regarding a binding of a PARP protein to a known PARP inhibitor; b) logic for designing a candidate PARP inhibitor based on the binding of the PARP protein and known PARP inhibitor; c) logic for determining an information regarding a binding of the PARP protein to the candidate PARP inhibitor; and d) logic for making a conclusion regarding the PARP inhibitory properties of the candidate PARP inhibitor based on the determination of step c).

Yet another aspect of the present invention relates to an electronic signal or carrier wave that is propagated over the internet between computers comprising a set of information for a general purpose computer having a user interface comprising a display unit, the set of information comprising a computer-readable storage medium containing a set of information for a general purpose computer having a user interface comprising a display unit, the set of information comprising: a) logic for inputting an information regarding a binding of a PARP protein to a known PARP inhibitor; b) logic for designing a candidate PARP inhibitor based on the binding of the PARP protein and known PARP inhibitor; c) logic for determining an information regarding a binding of the PARP protein to the candidate PARP inhibitor; and d) logic for making a conclusion regarding the PARP inhibitory properties of the candidate PARP inhibitor based on the determination of step c).

Another aspect of the present invention relates to a method of treating a disease comprising administering to a patient in need thereof an effective amount of at least one compound of formula I, its pharmaceutically acceptable salts or prodrugs thereof:

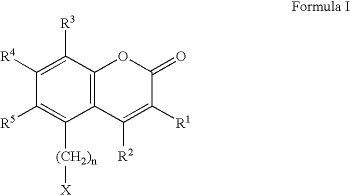

wherein n=0-10; R¹, R², R³, R⁴, R⁵ and X are independently selected from the group consisting of hydrogen, hydroxy, optionally substituted amine, carboxyl, ester, nitroso, nitro, halogen, optionally substituted (C₁-C₆) alkyl, optionally substituted (C₁-C₆) alkoxy, optionally substituted (C₃-C₇) cycloalkyl, optionally substituted (C₃-C₇) heterocyclic, and optionally substituted aryl; and wherein at least two of the R¹, R², R³, R⁴, and R⁵ substituents are always hydrogen. In some embodiments, n is 0, 1, 2, 3, 4, 5, 6, 7, 8, 9 or 10. In some embodiments, n is 1, 2, 3, 4, 5, 6, 7, 8, 9 or 10. In some embodiments, the halogen is selected from the group consisting of I, Br and Cl. In some embodiments, the halogen is Cl or Br. In some embodiments wherein R⁵ is amino, nitro or nitroso, n is 1, 2, 3, 4, 5, 6, 7, 8, 9 or 10. In some embodiments in which R⁵ is amino, nitro or nitroso, and n is 0, 1, 2, 3, 4, 5, 6, 7, 8, 9 or 10, X is optionally substituted (C₁-C₆) alkyl, optionally substituted (C₁-C₆) alkoxy, optionally substituted (C₃-C₇) cycloalkyl, optionally substituted (C₃-C₇) heterocyclic or optionally substituted aryl.

In some embodiments, the compound is of formula II or its pharmaceutically acceptable salts or prodrugs: 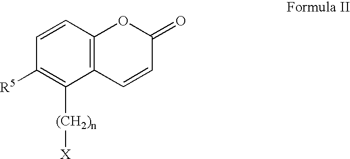
wherein R⁵ is selected from the group consisting of carboxyl, nitroso, and nitro; and X is selected from the group consisting of optionally substituted (C₁-C₇) alkyl, optionally substituted (C₁-C₆) alkoxy, optionally substituted (C₃-C₇) cycloalkyl, optionally substituted (C₃-C₇) heterocyclic, and optionally substituted aryl. In some preferred embodiments, the optionally substituted alkyl is substituted with a substituent selected from the group consisting of alkylamine, pyrrole, dihydropyrrole, or pyrrolidene.

In some preferred embodiments, the compound is of formula IIIa or its pharmaceutically acceptable salts or prodrugs:

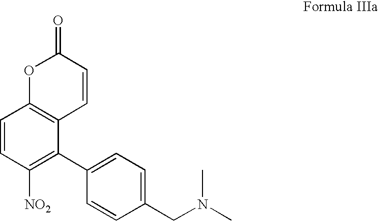

In some preferred embodiments, the compound is of formula IIIb or its pharmaceutically acceptable salts or prodrugs:

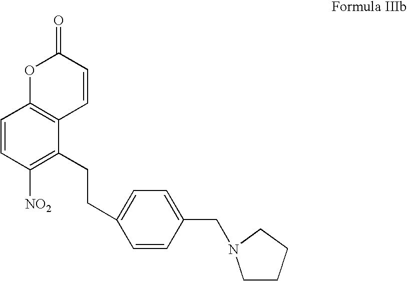

In some embodiments, the optionally substituted (C₃-C₇) heterocyclic is a five membered heterocyclic ring or a six membered heterocyclic ring. In some embodiments, the optionally substituted (C₃-C₇) heterocyclic contains at least one nitrogen. In some embodiments, the optionally substituted (C₃-C₇) heterocyclic is selected from the group consisting of azeridine, azetidine, pyrrole, dihydropyrrole, pyrrolidene, pyrazole, pyrazoline, pyrazolidine, imidazole, benzimidazole, triazole, tetrazole, oxazole, isoxazole, benzoxazole, oxadiazole, oxazoline, oxazolidine, thiazole, isothiazole, pyridine, dihydropyridine, tetrahydropyridine, quinazoline, pyrazine, pyrimidine, pyridazine, quinoline, isoquinoline, triazine, tetrazine, and piperazine. In some embodiments, the optionally substituted (C₃-C₇) heterocyclic is substituted with a substituent selected from the group consisting of optionally substituted (C₁-C₆) alkyl, optionally substituted (C₁-C₆) alkoxy, optionally substituted (C₃-C₇) cycloalkyl, optionally substituted (C₃-C₇) heterocyclic, and optionally substituted aryl.

In some preferred embodiments, the compound is of formula IIIc or its pharmaceutically acceptable salts or prodrugs:

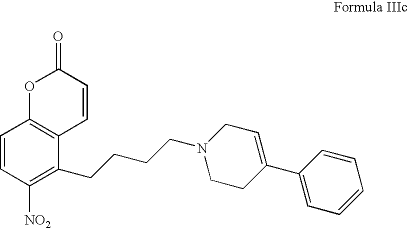

In some preferred embodiments, the compound is of formula IIId or its pharmaceutically acceptable salts or prodrugs:

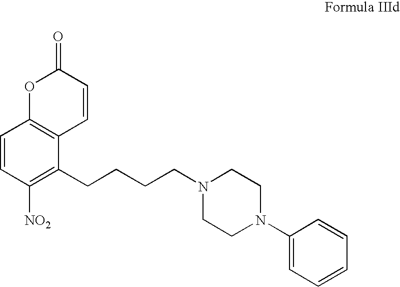

In some preferred embodiments, the compound is of formula IIIe or its pharmaceutically acceptable salts or prodrugs:

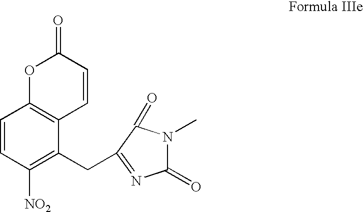

In some preferred embodiments, the compound is of formula IIIf or its pharmaceutically acceptable salts or prodrugs:

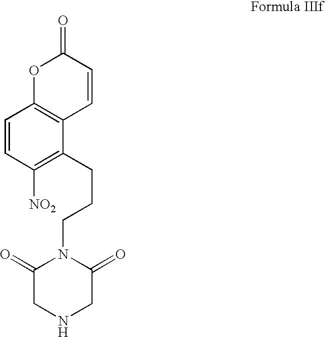

In some preferred embodiments, the treating comprises inhibiting a PARP protein. In some preferred embodiments, the disease is selected from the group consisting of cancer, inflammation, metabolic disease, CVS disease, CNS disease, disorder of hematolymphoid system, disorder of endocrine and neuroendocrine, disorder of urinary tract, disorder of respiratory system, disorder of female genital system, and disorder of male genital system.

Yet another aspect of the present invention relates to a compound of formula I, its pharmaceutically acceptable salts or prodrugs thereof: 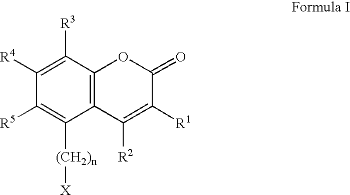
wherein n=0-10; R¹, R², R³, R⁴, R⁵ and X are independently selected from the group consisting of hydrogen, hydroxy, optionally substituted amine, carboxyl, ester, nitroso, nitro, halogen, optionally substituted (C₁-C₆) alkyl, optionally substituted (C₁-C₆) alkoxy, optionally substituted (C₃-C₇) cycloalkyl, optionally substituted (C₃-C₇) heterocyclic, and optionally substituted aryl; and wherein at least two of the R¹, R², R³, R⁴, and R⁵ substituents are always hydrogen. Preferably, the compound is a PARP inhibitor. In some embodiments, n is 0, 1, 2, 3, 4, 5, 6, 7, 8, 9 or 10. In some embodiments, n is 1, 2, 3, 4, 5, 6, 7, 8, 9 or 10. In some embodiments, the halogen is selected from the group consisting of I, Br and Cl. In some embodiments, the halogen is Cl or Br. In some embodiments wherein R⁵ is amino, nitro or nitroso, n is 1, 2, 3, 4, 5, 6, 7, 8, 9 or 10. In some embodiments in which R⁵ is amino, nitro or nitroso, and n is 0, 1, 2, 3, 4, 5, 6, 7, 8, 9 or 10, X is optionally substituted (C₁-C₆) alkyl, optionally substituted (C₁-C₆) alkoxy, optionally substituted (C₃-C₇) cycloalkyl, optionally substituted (C₃-C₇) heterocyclic or optionally substituted aryl.

In some preferred embodiments, the compound is of formula II or its pharmaceutically acceptable salts or prodrugs:

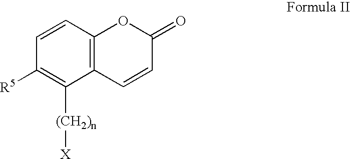

wherein R⁵ is selected from the group consisting of carboxyl, nitroso, and nitro; and X is selected from the group consisting of optionally substituted (C₁-C₇) alkyl, optionally substituted (C₁-C₆) alkoxy, optionally substituted (C₃-C₇) cycloalkyl, optionally substituted (C₃-C₇) heterocyclic, and optionally substituted aryl. Preferably, the compound is a PARP inhibitor.

In some embodiments, the compound is of formula III or its pharmaceutically acceptable salts or prodrugs:

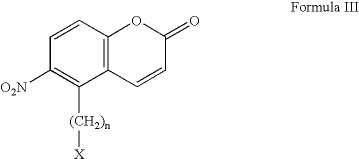

wherein n=0-10, and wherein X is selected from the group consisting of optionally substituted (C₃-C₇) cycloalkyl, optionally substituted (C₃-C₇) heterocyclic, and optionally substituted aryl. In some embodiments, the optionally substituted aryl is substituted with an optionally substituted alkyl. In some embodiments, the optionally substituted alkyl is substituted with a substituent selected from the group consisting of alkylamine, pyrrole, dihydropyrrole, or pyrrolidene. Preferably, the compound is a PARP inhibitor.

In some preferred embodiments, the compound is of formula IIIa or its pharmaceutically acceptable salts or prodrugs:

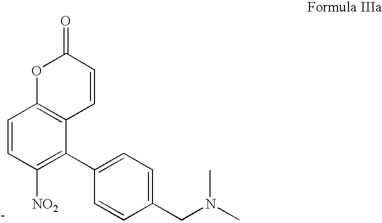

In some preferred embodiments, the compound is of formula IIIb or its pharmaceutically acceptable salts or prodrugs:

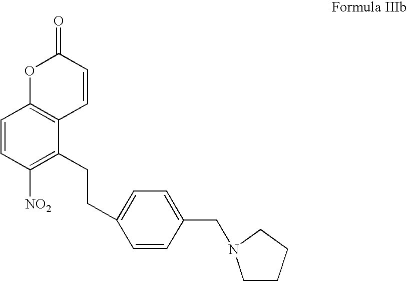

In some embodiments, the optionally substituted (C₃-C₇) heterocyclic is a five membered heterocyclic ring or a six membered heterocyclic ring. In some embodiments, the optionally substituted (C₃-C₇) heterocyclic contains at least one nitrogen. In some embodiments, the optionally substituted (C₃-C₇) heterocyclic is selected from the group consisting of azeridine, azetidine, pyrrole, dihydropyrrole, pyrrolidene, pyrazole, pyrazoline, pyrazolidine, imidazole, benzimidazole, triazole, tetrazole, oxazole, isoxazole, benzoxazole, oxadiazole, oxazoline, oxazolidine, thiazole, isothiazole, pyridine, dihydropyridine, tetrahydropyridine, quinazoline, pyrazine, pyrimidine, pyridazine, quinoline, isoquinoline, triazine, tetrazine, and piperazine.

In some embodiments, the optionally substituted (C₃-C₇) heterocyclic is substituted with a substituent selected from the group consisting of optionally substituted (C₁-C₆) alkyl, optionally substituted (C₁-C₆) alkoxy, optionally substituted (C₃-C₇) cycloalkyl, optionally substituted (C₃-C₇) heterocyclic, and optionally substituted aryl.

In some preferred embodiments, the compound is of formula IIIc or its pharmaceutically acceptable salts or prodrugs:

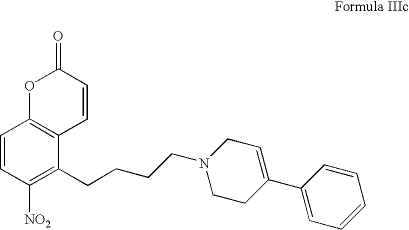

In some preferred embodiments, the compound is of formula IIId or its pharmaceutically acceptable salts or prodrugs:

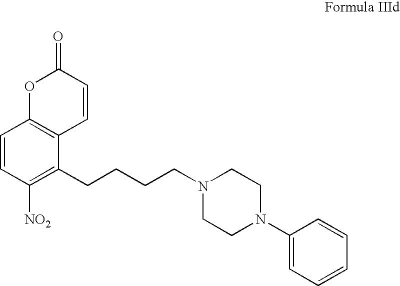

In some preferred embodiments, the compound is of formula IIIe or its pharmaceutically acceptable salts or prodrugs:

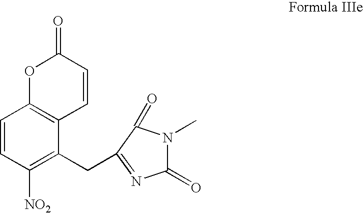

In some preferred embodiments, the compound is of formula IIIf or its pharmaceutically acceptable salts or prodrugs:

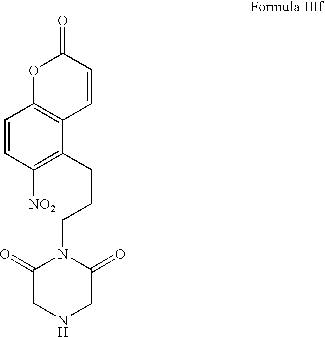

Yet another aspect of the invention relates to a compound comprising at least one structure selected from formula IIIa-f, its pharmaceutically acceptable salts or prodrugs thereof:

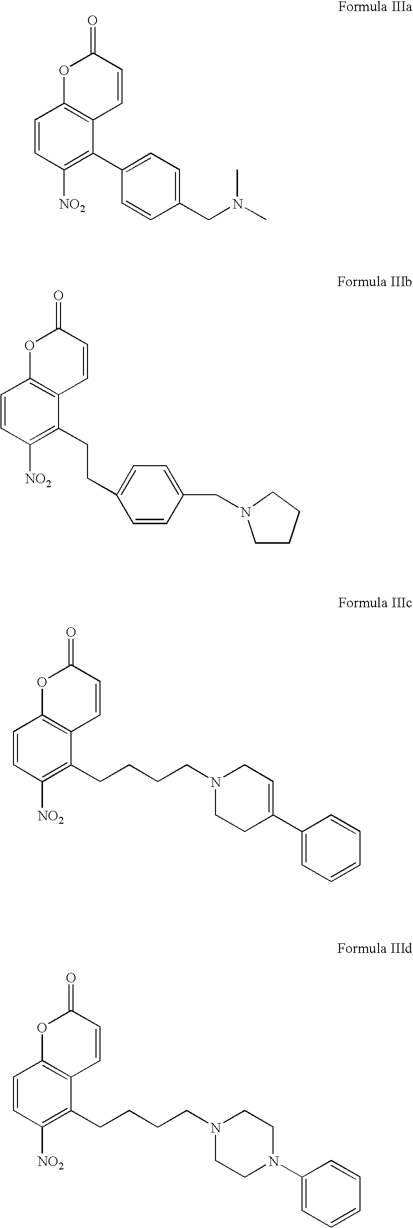

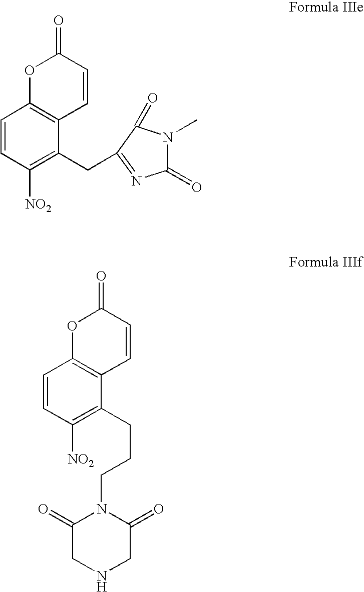

Another aspect of the present invention relates to a pharmaceutical composition comprising an effective amount of at least one compound as disclosed herein and a pharmaceutically acceptable carrier.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a flow chart showing the steps of the methods as disclosed herein.

FIG. 2 illustrates a computer for implementing selected operations associated with the methods disclosed herein.

FIG. 3 illustrates binding site of PARP1 (site blob generated by Molsoft PocketFinder). FIG. 3 illustrates the binding modes of the two compounds, 3-(4-chlorophenyl)quinoxaline-5-carboxamide and 5-fluoro-1-[4-(4-phenyl-3,6-dihydropyridin-1(butyl]quinazoline-2,4(1h,3h)-dione within the identified pocket of PARP 1 protein (X-ray structures 1WOK and 1UK1).

FIG. 4 illustrates 5-iodo-6-nitrocoumarin docked into the inhibitor binding pocket on PARP-1 where various other inhibitors with known complex x-ray structures are superimposed on the structure of 5-iodo-6-nitrocoumarin.

FIG. 5 illustrates a detailed view of 5-iodo-6-nitrocoumarin docking as compared to the x-ray structure of bound 3,4-dihydro-5-methyl-isoquinolinone.

### DETAILED DESCRIPTION OF THE INVENTION

### Definitions

The term, “aryl” refers to optionally substituted mono- or bicyclic aromatic rings containing only carbon atoms. The term can also include phenyl group fused to a monocyclic cycloalkyl or monocyclic cycloheteroalkyl group in which the point of attachment is on an aromatic portion. Examples of aryl groups include, e.g., phenyl, naphthyl, indanyl, indenyl, tetrahydronaphthyl, 2,3-dihydrobenzofuranyl, dihydrobenzopyranyl, 1,4-benzodioxanyl, and the like.

The term, “heterocyclic” refers to an optionally substituted mono- or bicyclic aromatic ring containing at least one heteroatom (an atom other than carbon), such as N, O and S, with each ring containing about 5 to about 6 atoms. Examples of heterocyclic groups include, e.g., pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, furo(2,3-b)pyridyl, quinolyl, indolyl, isoquinolyl, and the like.

The term, “computer system” as used herein, means the hardware means, software means and data storage means used to perform method of the present invention. Preferably, the computer system is used to analyze atomic coordinate data. The minimum hardware means of the computer-based systems of the present invention comprises a central processing unit (CPU), input means, output means and data storage means. Desirably a monitor is provided to visualize the structure data. The computer can be a stand-alone, or connected to a network and/or shared server. The data storage means can be RAM or means for accessing computer readable media of the invention.

The term, “computer readable media” as used herein, means any media which can be read and accessed by a computer, for example, the media is suitable for use in the above-mentioned computer system. The media include, but are not limited to: magnetic storage media such as floppy discs, hard disc storage medium and magnetic tape; optical storage media such as optical discs or CD-ROM; electrical storage media such as RAM and ROM; and hybrids of these categories such as magnetic/optical storage media.

The term “inhibit” or its grammatical equivalent, such as “inhibitory,” is not intended to require complete reduction in biological activity, preferably, PARP activity. Such reduction is preferably by at least about 50%, at least about 75%, at least about 90%, and more preferably by at least about 95% of the activity of the molecule in the absence of the inhibitory effect, e.g., in the absence of a PARP inhibitor as disclosed in the invention. Most preferably, the term refers to an observable or measurable reduction in activity. In treatment scenarios, preferably the inhibition is sufficient to produce a therapeutic and/or prophylactic benefit in the condition being treated.

The term “model” or its grammatical equivalents, such as, “modeling” as used herein, means the quantitative and qualitative analysis of molecular structure and/or function based on atomic structural information and interaction models.

The term “modeling” includes for example, conventional numeric-based molecular dynamic and energy minimization models, interactive computer graphic models, modified molecular mechanics models, distance geometry and other structure-based constraint models.

The term “pharmaceutically acceptable salt” as used herein, means those salts which retain the biological effectiveness and properties of the compounds of the present invention, and which are not biologically or otherwise undesirable.

The term “substituted” includes single or multiple degrees of substitution by a named substituent.

The term “candidate PARP inhibitor” as used herein, means any compound which is potentially capable of associating with PARP protein, and/or inhibiting PARP protein activity and/or the ability of PARP protein to interact with another molecule. The candidate compound can be designed or obtained from a library of compounds which can comprise peptides, as well as other compounds, such as small organic molecules and particularly new lead compounds. By way of example, the candidate compound can be a natural substance, a biological macromolecule, or an extract made from biological materials such as bacteria, fungi, or animal (particularly mammalian) cells or tissues, an organic or an inorganic molecule, a synthetic test compound, a semi-synthetic test compound, a carbohydrate, a monosaccharide, an oligosaccharide or polysaccharide, a glycolipid, a glycopeptide, a saponin, a heterocyclic compound, a structural or functional mimetic, a peptide, a peptidomimetic, a derivatized test compound, a peptide cleaved from a whole protein, or a peptides synthesized synthetically (such as, by way of example, either using a peptide synthesizer or by recombinant techniques or combinations thereof), a recombinant test compound, a natural or a non-natural test compound, a fusion protein or equivalent thereof and mutants, derivatives or combinations thereof.

The term “treating” or its grammatical equivalents as used herein, means achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder. For prophylactic benefit, the compositions can be administered to a patient at risk of developing a particular disease, or to a patient reporting one or more of the physiological symptoms of a disease, even though a diagnosis of this disease may not have been made.

### Methods for Designing a PARP Inhibitor

One aspect of the present invention relates to methods for designing a PARP inhibitor. In some preferred embodiments, the designing comprises using computer modeling techniques. In particular, the present invention relates to a computer-assisted method for design of a PARP inhibitor comprising: a) determining an interaction between a PARP protein and a known PARP inhibitor by evaluating a binding of the PARP protein to the known PARP inhibitor; b) based on the interaction, designing a candidate PARP inhibitor; c) determining an interaction between the PARP protein and the candidate PARP inhibitor by evaluating a binding of the PARP protein to the candidate PARP inhibitor; and d) concluding that the candidate PARP inhibitor inhibits the PARP protein wherein the conclusion is based on the interaction of step c).

In some preferred embodiments, a three-dimensional structure comprising a binding domain of the PARP protein and a three-dimensional structure of the known PARP inhibitor is used for determining an interaction between the PARP protein and the known PARP inhibitor. Preferably, the PARP protein is PARP 1 protein. In still preferred embodiments, a three dimensional structure of a binding domain of a PARP protein is modeled using a crystal of PARP protein through x-ray crystallographic techniques. A three dimensional structure of a known PARP inhibitor is modeled based on techniques known in the art. The three dimensional structure of a known PARP inhibitor is allowed to interact with the three dimensional structure of the binding domain of the PARP protein. Various PARP inhibitors are known in the art and are within the scope of the present invention. Some of the examples of the known PARP inhibitors include, but are not limited to, iodonitocoumarin, 5-iodo-6-nitrocoumarin, 3,4-dihydro-5-methyl-isoquinolinone, 4-amino-1,8-naphthalimide, 3 methoxybenzamide, 8-hydroxy-2-methyl-3-hydro-quinazolin-4-one, 2-{3-[4-(4-fluorophenyl)-3,6-dihydro-1(2h)-pyridinyl]propyl}-8-methyl-4(3h)-quinazolinone, 5-fluoro-1-[4-(4-phenyl-3,6-dihydropyridin-1 (butyl]quinazoline-2,4(1h,3h)-dione, 3-(4-chlorophenyl)quinoxaline-5-carboxamide, and 2-(3′-methoxyphenyl)benzimidazole-4-carboxam. In some preferred embodiments of the present invention, the known PARP inhibitor is 5-iodo-6-nitrocoumarin.

An interaction between the PARP protein and the known PARP inhibitor is determined based on an evaluation of a three dimensional structure of a binding domain of a PARP protein bound to the known PARP inhibitor. The evaluation can comprise evaluation of one or more of steric interactions, van der Waals interactions, electrostatic interactions, solvation interactions, charge interactions, covalent bonding interactions, non-covalent bonding interactions, entropically favorable interactions, or enthalpically favorable interactions. The techniques for the evaluation of such interactions between the enzyme and the drug are well known in the art and are well within the scope of the present invention.

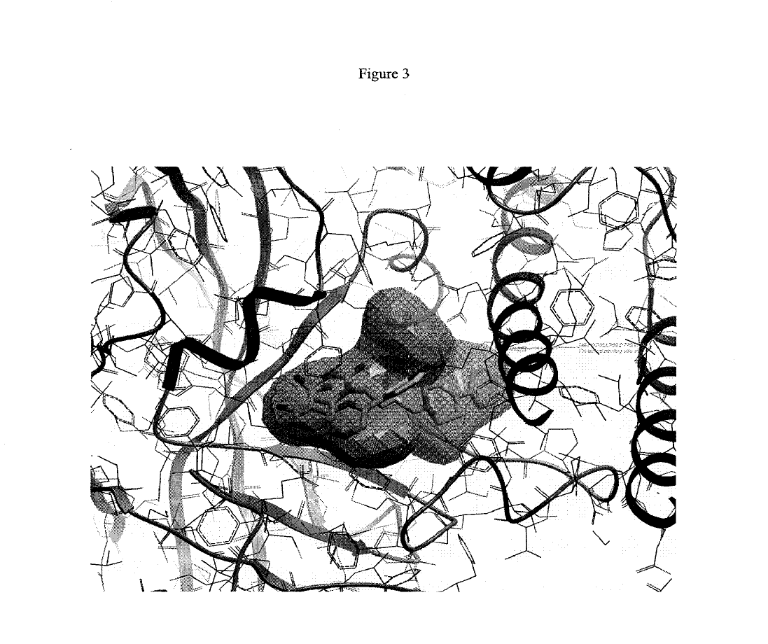

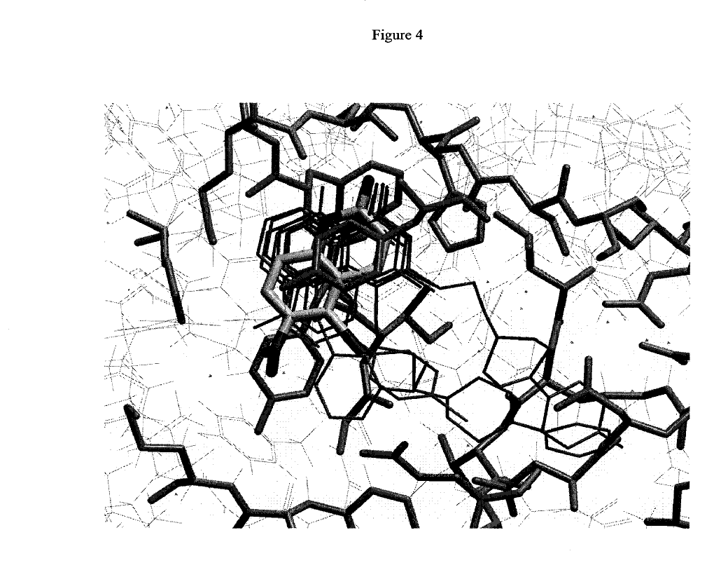

For example, FIG. 3 illustrates binding site of PARP1 (generated by Molsoft PocketFinder). FIG. 3 illustrates the binding modes of the two PARP inhibitors such as, 3-(4-chlorophenyl)quinoxaline-5-carboxamide and 5-fluoro-1-[4-(4-phenyl-3,6-dihydropyridin-1(butyl]quinazoline-2,4(1h,3h)-dione within the identified pocket of PARP 1 protein (X-ray structures 1WOK and 1UK1). FIG. 4 illustrates a 5-iodo-6-nitrocoumarin docked into the inhibitor binding pocket on PARP-1. Various inhibitors with known x-ray structures are superimposed on 5-iodo-6-nitrocoumarin. Lactone ring of 5-iodo-6-nitrocoumarin largely overlaps lactams of the known inhibitors, thereby preserving the interactions of the carbonyl oxygen.

Based on the evaluation of the binding of the known PARP inhibitor with the PARP protein, a candidate PARP inhibitor can be designed. Preferably, the candidate PARP inhibitor is designed using computer modeling. In some preferred embodiments, the candidate PARP inhibitor is an analog of the known PARP inhibitor. In still further preferred embodiments, the candidate PARP inhibitor is an analog of the 5-iodo-6-nitrocoumarin. A candidate PARP inhibitor can be designed in such a way that it fits equally or more efficiently in the binding domain of the PARP protein as compared to the known PARP inhibitor.

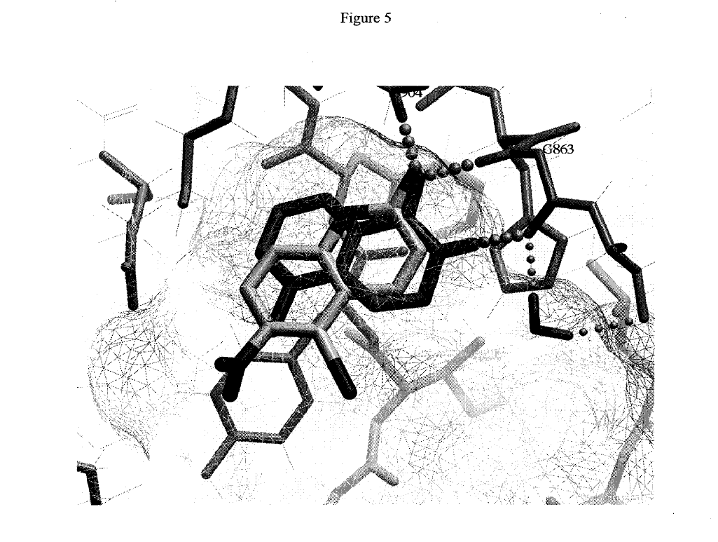

For example, FIG. 5 illustrates a detailed view of the 5-iodo-6-nitrocoumarin docking as compared to the x-ray structure of bound 3,4-dihydro-5-methyl-isoquinolinone. Isoquinolinone forms an extra hydrogen bond using a polar proton on its lactam nitrogen. This extra hydrogen bond is missing in the iodocoumarin due to the absence of a lactam nitrogen. The backbone carbonyl oxygen (G863), however, remains coordinated by a bound water molecule, and likely still forms a weaker hydrogen bond with an olefinic hydrogen of the coumarin. At the same time, nitro group and iodine provide additional van der Waals interactions. Hence, the candidate PARP inhibitor can comprise one or more modifications to the known PARP inhibitor in such a way that the candidate PARP inhibitor fits well within the binding domain of the PARP protein. For example, the iodo or the nitro group in the 5-iodo-6-nitrocoumarin can be replaced with another group in such a way that the resulting candidate PARP inhibitor provides more interaction with the binding domain of the PARP protein as compared to the 5-iodo-6-nitrocoumarin.

In some preferred embodiments of the present invention, an iodo group of the 5-iodo-6-nitrocoumarin is replaced with another group in a candidate PARP inhibitor. Preferably, the other group that replaces iodo group in the known PARP inhibitor improves the solubility of the candidate PARP inhibitor by virtue of having at least one nitrogen atom. Hence, the other group can impart hydrophilic characteristics to the candidate PARP inhibitor. More preferably, the other group that replaces iodo group in the known PARP inhibitor improves the binding of the candidate PARP inhibitor with the binding domain of the PARP protein.

After the designing of the candidate PARP inhibitor, an interaction between the PARP protein and the candidate PARP inhibitor can be determined based on an evaluation of the three dimensional structure of the binding domain of the PARP protein bound to the candidate PARP inhibitor. The evaluation can comprise evaluation of one or more of steric interactions, van der Waals interactions, electrostatic interactions, solvation interactions, charge interactions, covalent bonding interactions, non-covalent bonding interactions, entropically favorable interactions, or enthalpically favorable interactions. Based on the evaluation a conclusion can be made regarding the candidate PARP inhibitor's ability to inhibit the PARP protein.

Alternatively, the PARP protein can be co-crystallized with a candidate PARP inhibitor in order to provide a crystal suitable for determining the structure of the complex. A crystal of the PARP protein can be soaked in a solution containing the candidate PARP inhibitor in order to form co-crystals by diffusion of the candidate PARP inhibitor into the crystal of the PARP protein. In some embodiments, the structure of the PARP protein obtained in the presence and absence of the candidate PARP inhibitor can be compared to determine structural information about the PARP protein, identification of druggable regions of the PARP protein and/or determine the interaction between the candidate PARP inhibitor and the PARP protein.

The present invention further relates to methods for synthesizing the candidate PARP inhibitors by conventional synthetic chemistry techniques. These techniques are known in the art and are within the scope of the present invention. The present invention further relates to assessing the bioactivity, such as PARP inhibiting activity, of the synthesized PARP inhibitor compounds. The assay techniques for assessing the bioactivity of the candidate PARP inhibitor are well known in the art and are within the scope of the present invention. Another aspect of the present invention relates to providing methods of treatment of a disease using the PARP inhibitors. Preferably, the disease is a PARP related condition.

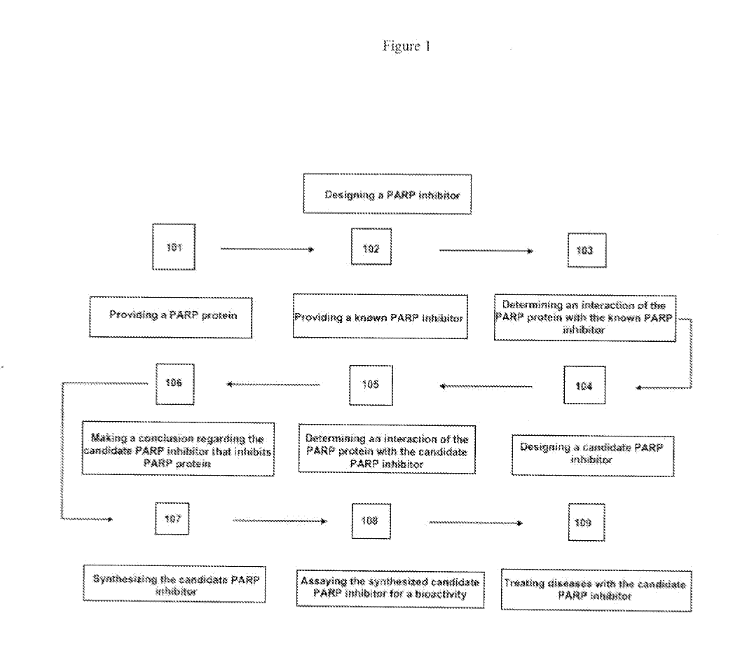

The steps for some of the embodiments of the present invention are depicted in FIG. 1. Without limiting the scope of the present invention, the steps can be performed independent of each other or one after the other. One or more steps can be skipped in the methods of the present invention. A PARP protein is provided at step 101. In some preferred embodiments, the PARP protein is PARP 1 protein. In still some preferred embodiments, a three dimensional structure of the PARP protein is provided. Preferably, the three dimensional structure of the PARP protein is modeled from a crystal of PARP protein using x-ray crystallography.

A known PARP inhibitor is provided at step 102. In some preferred embodiments, a three dimensional structure of the known PARP inhibitor is provided. Preferably, the three dimensional structure of the known PARP inhibitor is provided by a computer modeling technique. An interaction between the PARP protein and the known PARP inhibitor is determined based on the evaluation of the binding of the PARP protein to the known PARP inhibitor at step 103.

Based on the evaluation, a candidate PARP inhibitor is designed at step 104. Preferably, the candidate PARP inhibitor is designed by computer modeling. An interaction between the PARP protein and the candidate PARP inhibitor is determined based on the evaluation of the binding of the PARP protein to the candidate PARP inhibitor at step 105. Based on this evaluation, a conclusion is made regarding a candidate PARP inhibitor that inhibits PARP protein at step 106. Further, the candidate PARP inhibitor that inhibits PARP protein is chemically synthesized at step 107. The chemically synthesized candidate PARP inhibitor is assayed for its bioactivity, preferably, PARP inhibiting activity at step 108. The candidate PARP inhibitor that inhibits PARP protein is used for treating diseases at step 109. It shall be understood that the invention includes other methods not explicitly set forth herein.

Poly (ADP-Ribose) Polymerase (PARP)

The poly (ADP-ribose) polymerase (PARP) is also known as poly (ADP-ribose) synthase and poly ADP-ribosyltransferase. PARP catalyzes the formation of poly (ADP-ribose) polymers which can attach to nuclear proteins (as well as to itself) and thereby modify the activities of those proteins. The enzyme plays a role in enhancing DNA repair, but more fundamentally there are indications that it plays a major role in regulating chromatin in the nuclei (for review see: D. D'Amours et al. “Poly (ADP-ribosylation reactions in the regulation of nuclear functions,” Biochem. J. 342: 249-268 (1999)).

More than 15 members of the PARP family of genes are present in the mammalian genome. PARP family proteins and poly(ADP-ribose) glycohydrolase (PARG), which degrades poly(ADP-ribose) to ADP-ribose, could be involved in a variety of cell regulatory functions including DNA damage response and transcriptional regulation and can be related to carcinogenesis and the biology of cancer in many respects.

Several PARP family proteins have been identified. Tankyrase has been found as an interacting protein of telomere regulatory factor 1 (TRF-1) and is involved in telomere regulation. Vault PARP (VPARP) is a component in the vault complex, which acts as a nuclear-cytoplasmic transporter. PARP-2, PARP-3 and 2,3,7,8-tetrachlorodibenzo-p-dioxin inducible PARP (TiPARP) have also been identified. Therefore, poly (ADP-ribose) metabolism could be related to a variety of cell regulatory functions.

The most studied member of this gene family is PARP-1. The PARP-1 gene product is expressed at high levels in the nuclei of cells and is dependent upon DNA damage for activation. Without being bound by any theory, it is believed that PARP-1 binds to DNA single or double stranded breaks through an amino terminal DNA binding domain. The binding activates the carboxy terminal catalytic domain and results in the formation of polymers of ADP-ribose on target molecules. PARP-1 is itself a target of poly ADP-ribosylation by virtue of a centrally located automodification domain. The ribosylation of PARP-1 causes dissociation of the PARP-1 molecules from the DNA. The entire process of binding, ribosylation, and dissociation occurs very rapidly. It has been suggested that this transient binding of PARP-1 to sites of DNA damage results in the recruitment of DNA repair machinery or can act to suppress the recombination long enough for the recruitment of repair machinery.

The source of ADP-ribose for the PARP reaction is nicotinamide adenosine dinucleotide (NAD). NAD is synthesized in cells from cellular ATP stores and thus high levels of activation of PARP activity can rapidly lead to depletion of cellular energy stores. It has been demonstrated that induction of PARP activity can lead to cell death that is correlated with depletion of cellular NAD and ATP pools. PARP activity is induced in many instances of oxidative stress or during inflammation. For example, during reperfusion of ischemic tissues reactive nitric oxide is generated and nitric oxide results in the generation of additional reactive oxygen species including hydrogen peroxide, peroxynitrate and hydroxyl radical. These latter species can directly damage DNA and the resulting damage induces activation of PARP activity. Frequently, it appears that sufficient activation of PARP activity occurs such that the cellular energy stores are depleted and the cell dies. A similar mechanism is believed to operate during inflammation when endothelial cells and pro-inflammatory cells synthesize nitric oxide which results in oxidative DNA damage in surrounding cells and the subsequent activation of PARP activity. The cell death that results from PARP activation is believed to be a major contributing factor in the extent of tissue damage that results from ischemia-reperfusion injury or from inflammation.

Inhibition of PARP activity can be potentially useful in the treatment of cancer. De-inhibition of the DNAase (by PARP-1 inhibition) can initiate DNA breakdown that is specific for cancer cells and to only induce apoptosis in cancer cells. Small PARP molecule inhibitors can sensitize treated tumor cell lines to killing by ionizing radiation and by some DNA damaging chemotherapeutic drugs. A monotherapy by PARP inhibitors or a combination therapy of PARP inhibitors with a chemotherapeutic agent or radiation can be an effective treatment. Combination therapy with a chemotherapeutic can induce tumor regression at concentrations of the chemotherapeutic that are ineffective by themselves.

Binding Domains of PARP

In some embodiments of the present invention, the known PARP inhibitor and/or the candidate PARP inhibitor interact with a binding domain of the PARP protein. Preferably, the binding domain is a catalytic domain.

PARP-1 comprises an N-terminal DNA binding domain, an automodification domain and a C-terminal catalytic domain and various cellular proteins interact with PARP-1. The N-terminal DNA binding domain contains two zinc finger motifs. Transcription enhancer factor-1 (TEF-1), retinoid X receptor α, DNA polymerase α, X-ray repair cross-complementing factor-1 (XRCC1) and PARP-1 itself interacts with PARP-1 in this domain. The automodification domain contains a BRCT motif, one of the protein-protein interaction modules. This motif is originally found in the C-terminus of BRCA1 (breast cancer susceptibility protein 1) and is present in various proteins related to DNA repair, recombination and cell-cycle checkpoint control. POU-homeodomain-containing octamer transcription factor-1 (Oct-1), Yin Yang (YY)1 and ubiquitin-conjugating enzyme 9 (ubc9) could interact with this BRCT motif in PARP-1.

PARP-2 lacks the N-terminal tandem zinc fingers and BRCT domain of PARP-1, which are replaced by a small highly basic N-terminal DNA-binding domain, with the E domain acting both as a dimerization and automodification domain, but shares the C-terminal catalytic domain, which is the unifying feature of the wider PARP family. (See Oliver et al., Nucleic Acids Research, Vol. 32, No. 2, 456-464 (2004)).

Crystal Structure of PARP

Examples of methods for determining structure information of PARP protein or PARP bound with a inhibitor include: 1) mass spectrometry to determine one or more properties of a protein, including primary sequence, post translation modification, protein-small molecule interaction, or protein-protein interaction ability; 2) NMR, including ID NMR, multidimensional NMR, and multinuclear NMR, such as ¹⁵N/¹H HSQC spectra, to determine one or more properties of a protein including three dimensional structure, conformational states, aggregation level, state of protein folding or unfolding, or the dynamic properties of the protein; and 3) x-ray crystallography to determine one or more properties of a protein, including three dimensional structure, diffraction of its crystal form or its space group. The present invention preferably uses x-ray crystallography to determine the structural characteristics of the PARP protein. In particular, x-ray diffraction of a crystallized form of the PARP protein can be used to determine the three dimensional structure of the PARP protein.

Crystals of PARP protein can be produced or grown by a number of techniques including batch crystallization, vapor diffusion (either by sitting drop or hanging drop), soaking, and by microdialysis. Seeding of the crystals in some instances can be required to obtain x-ray quality crystals. Standard micro and/or macro seeding of crystals can be used. The crystal can diffract x-rays for the determination of the atomic coordinates of the PARP protein to a resolution greater than 5.0 Angstroms, alternatively greater than 3.0 Angstroms, or alternatively greater than 2.0 Angstroms.

Crystals can be grown from a solution containing a purified PARP protein, or a fragment thereof (e.g., a stable domain), by a variety of conventional processes (McPherson, 1982 John Wiley, New York; McPherson, 1990, Eur. J. Biochem. 189: 1-23; Webber. 1991, Adv. Protein Chem. 41:1-36). In some embodiments, native crystals of the PARP protein can be grown by adding precipitants to the concentrated solution of the PARP protein. The precipitants can be added at a concentration just below that necessary to precipitate the PARP protein. Water can be removed by controlled evaporation to produce precipitating conditions, which are maintained until crystal growth ceases. The formation of crystals can depend on various factors including pH, temperature, PARP protein concentration, the nature of the solvent and precipitant, as well as the presence of added ions or ligands to the PARP protein. In addition, the sequence of the PARP protein being crystallized can have an affect on the success of obtaining crystals. Many routine crystallization experiments can be needed to screen all these factors for the few combinations that might give crystal suitable for x-ray diffraction analysis. Crystallization robots can automate and speed up the work of reproducibly setting up large number of crystallization experiments. Once the conditions for growing the crystal are optimized, variations of the condition can be systematically screened in order to find the set of conditions which allow the growth of sufficiently large, single, well ordered crystals. In some embodiments, the PARP protein can be co-crystallized with a compound that stabilizes the PARP protein.

Before the data collection, the PARP protein crystal can be frozen to protect it from radiation damage. A number of different cryo-protectants can be used to assist in freezing the crystal, such as methyl pentanediol (MPD), isopropanol, ethylene glycol, glycerol, formate, citrate, mineral oil, or a low-molecular-weight polyethylene glycol (PEG). As an alternative to freezing the crystal, the crystal can also be used for diffraction experiments performed at temperatures above the freezing point of the solution. In these instances, the crystal can be protected from drying out by placing it in a narrow capillary of a suitable material (generally glass or quartz) with some of the crystal growth solution included in order to maintain vapor pressure.

X-ray diffraction results can be recorded by a number of ways know to one of skill in the art. Collection of X-ray diffraction patterns are well known by those skilled in the art and are within the scope of the present invention. Modeling of the three dimensional structure of the PARP protein can be accomplished by either the crystallographer using a computer graphics program such as TURBO or O (Jones, T A. et al., Acta Crystallogr. A47, 100-119, 1991) or, under suitable circumstances, by using a fully automated model building program, such as WARP (Anastassis et al. Nature Structural Biology, May 1999 Volume 6 Number 5 pp 458-463) or MAID (Levitt, D. G., Acta Crystallogr. D 2001 V57: 1013-9). This structure can be used to calculate model-derived diffraction amplitudes and phases.

The three dimensional structure of the crystal of the PARP protein can be modeled using molecular replacement. The term “molecular replacement” refers to a method that involves generating a preliminary model of a molecule or complex whose structure coordinates are unknown, by orienting and positioning a molecule whose structure coordinates are known within the unit cell of the unknown crystal, so as best to account for the observed diffraction pattern of the unknown crystal. Phases can then be calculated from this model and combined with the observed amplitudes to give an approximate Fourier synthesis of the structure whose coordinates are unknown. This, in turn, can be subject to any of the several forms of refinement to provide a final, more accurate structure of the unknown crystal.

Homology modeling (also known as comparative modeling or knowledge-based modeling) methods can also be used to develop a three dimensional structure of the PARP protein. The method utilizes a computer model of a known protein, a computer representation of the amino acid sequence of the polypeptide (e.g., PARP protein) with an unknown structure, and standard computer representations of the structures of amino acids. This method is well known to those skilled in the art (Greer, 1985, Science 228, 1055; Bundell et al 1988, Eur. J. Biochem. 172, 513).

A three dimensional structure of the PARP protein can be described by the set of atoms that best predict the observed diffraction data. Files can be created for the structure that defines each atom by its chemical identity, spatial coordinates in three dimensions, root mean squared deviation from the mean observed position and fractional occupancy of the observed position. Hydrogen bonds and other atomic interactions, both within the protein and to bound ligands, can be identified. A model can represent the secondary, tertiary and/or quaternary structure of the PARP protein. The model itself can be in two or three dimensions.

It is known in the art that a set of structure coordinates for a protein, complex or a portion thereof, is a relative set of points that define a shape in three dimensions. Thus, it is possible that an entirely different set of coordinates could define a similar or identical shape. Moreover, slight variations in the individual coordinates can have little effect on overall shape. Such variations in coordinates can be generated because of mathematical manipulations of the structure coordinates. For example, structure coordinates could be manipulated by crystallographic permutations of the structure coordinates, fractionalization of the structure coordinates, integer additions or subtractions to sets of the structure coordinates, inversion of the structure coordinates or any combination of the above.

The three-dimensional structure of the PARP protein, a known PARP inhibitor, a candidate PARP inhibitor, or a PARP protein bound to a known PARP inhibitor or a candidate PARP inhibitor (PARP protein-PARP inhibitor complex), can be determined by conventional means as described above or as known in the art. The structure factors from the three-dimensional structure coordinates of PARP protein can be utilized to aid the structure determination of the PARP protein-PARP inhibitor complex. Structure factors include mathematical expressions derived from three-dimensional structure coordinates of the PARP protein. These mathematical expressions include, for example, amplitude and phase information. The three-dimensional structure of the PARP protein, a known PARP inhibitor, a candidate PARP inhibitor or a PARP protein-PARP inhibitor complex can be determined using molecular replacement analysis. This analysis utilizes a known three-dimensional structure as a search model to determine the structure of a closely related PARP protein, a known PARP inhibitor, a candidate PARP inhibitor or a PARP protein-PARP inhibitor complex.

In some embodiments, the PARP protein can be soluble, purified and/or isolated PARP protein which can optionally comprise a tag or label to facilitate expression, purification and/or structural or functional characterization. In some embodiments, a PARP protein which is used in accordance with the methods of the invention is labeled with an isotopic label to facilitate its detection and or structural characterization using nuclear magnetic resonance or another applicable technique. Exemplary isotopic labels include radioisotopic labels such as, for example, potassium-40 (⁴⁰K), carbon-14 (¹⁴C), tritium (³H), sulfur-35 (³⁵S), phosphorus-32 (³²P), technetium-99m (⁹⁹ mTc), thallium-201 (²⁰¹Tl), gallium-67 (⁶⁷Ga), indium-111 (¹¹¹In), iodine-123 (¹²³I), iodine-131 (¹³¹I), yttrium-90 (⁹⁰Y), samarium-153 (¹⁵³Sm), rhenium-186 (¹⁸⁶Re), rhenium-188 (¹⁸⁸Re), dysprosium-165 (¹⁶⁵Dy) and holmium-166 (¹⁶⁶Ho). The isotopic label can also be an atom with non zero nuclear spin, including, for example, hydrogen-1 (¹H), hydrogen-2 (²H), hydrogen-3 (³H), phosphorous-31 (³¹P), sodium-23 (²³Na), nitrogen-14 (¹⁴N), nitrogen-15 (¹⁵N), carbon-13 (¹³C) and fluorine-19 (¹⁹F).

In certain embodiments, the PARP protein is uniformly labeled with an isotopic label, for example, wherein at least about 50%, 70%, 80%, 90%, 95%, or 98% of the possible labels in the PARP protein are labeled, e.g., wherein at least about 50%, 70%, 80%, 90%, 95%, or 98% of the nitrogen atoms in the PARP protein are ¹⁵N, and/or wherein at least about 50%, 70%, 80%, 90%, 95%, or 98% of the carbon atoms in the PARP protein are ¹³C, and/or wherein at least about 50%, 70%, 80%, 90%, 95%, or 98% of the hydrogen atoms in the PARP protein are ²H. In other embodiments, the isotopic label is located in one or more specific locations within the PARP protein. The invention also encompasses the embodiment wherein a single PARP protein comprises two or more different isotopic labels, for example, the PARP protein comprises both ¹⁵N and ¹³C labeling.

In yet another embodiment, the PARP protein which can be used in accordance with the methods of the invention is labeled to facilitate structural characterization using x-ray crystallography or another applicable technique. Exemplary labels include heavy atom labels such as, for example, cobalt, selenium, krypton, bromine, strontium, molybdenum, ruthenium, rhodium, palladium, silver, cadmium, tin, iodine, xenon, barium, lanthanum, cerium, praseodymium, neodymium, samarium, europium, gadolinium, terbium, dysprosium, holmium, erbium, thulium, ytterbium, lutetium, tantalum, tungsten, rhenium, osmium, iridium, platinum, gold, mercury, thallium, lead, thorium and uranium.

Designing a PARP Inhibitor

Designing as disclosed in the present invention involves designing a chemical substance, particularly a candidate PARP inhibitor that interacts in some way with receptors or binding domains of the PARP protein. Preferably, the PARP protein is PARP 1 protein. Typically, for a drug to effectively interact with the binding domains of the PARP protein, it can be necessary that the three-dimensional shape (“conformation”) of PARP protein assumes a compatible conformation that allows the drug and the binding domain of the PARP protein to fit and bind together in a way that produces a desired result. Preferably, the desired result is an efficient binding of the drug with the PARP protein resulting in an inhibition of the PARP activity. In such instance, the complex shape or conformation of the binding domain of the PARP protein can be compared to a “lock”, and the corresponding requisite shape or conformation of the drug as a “key” that unlocks (i.e., produces the desired result within) the binding domain of the PARP protein. This “lock-and-key” analogy emphasizes that only a properly conformed key (drug patterned thereafter) is able to fit within the lock (the binding domain of the PARP protein) in order to “unlock” it (produce a desired result). Further, even if the key fits in the lock, it must have the proper composition in order for it to perform its function. That is, the drug contains the elements in the spatial arrangement and position in order to properly bind with the binding domain of the PARP protein. The design as disclosed herein can include knowing or predicting the conformation of the binding domain of the PARP protein, and also controlling and/or predicting the conformation of the drug, i.e., a candidate PARP inhibitor that is to interact with the binding domain of the PARP protein.

Determination of the binding domain of the PARP protein, and in particular the recognition of the role of catalytic domain can help in identifying binding of the PARP inhibitors in the binding domain of the PARP protein. A known PARP inhibitor typically can be used to evaluate its binding with the binding domain of the PARP protein. Based on this evaluation, computational techniques for drug design are used to design candidate PARP inhibitors based on the structure of a known PARP inhibitor. For example, automated ligand-receptor docking programs which require accurate information on the atomic coordinates of target receptors are used to design candidate PARP inhibitors. The candidate PARP inhibitors can be designed de novo or can be analogs of a known PARP inhibitor. Preferably, the candidate PARP inhibitor is designed based on a known PARP inhibitor. More preferably, the candidate PARP inhibitor is an analog of 5-iodo-6-nitrocoumarin. Alternatively, the PARP inhibitors can be synthesized and formed into a complex with PARP protein, and the complex can then be analyzed by x-ray crystallography to identify the actual position of the bound PARP inhibitor. The structure and/or functional groups of the PARP inhibitor can then be adjusted, if necessary, in view of the results of the x-ray analysis, and the synthesis and analysis sequence repeated until an optimized PARP inhibitor is obtained.

The designing of the candidate PARP inhibitor can involve computer-based in silico screening of compound databases (such as the Cambridge structural database) with the aim of identifying compounds which interact with the binding cavity or sites of the target PARP protein. Screening selection criteria can be based on pharmacokinetic properties such as metabolic stability and toxicity. Determination of the mechanism of the PARP inhibition allows the architecture and the chemical nature of the PARP binding site to be better defined, which in turn allows the geometric and functional constraints of a substituent on the candidate PARP inhibitor to be derived more accurately. The substituent can be a type of virtual 3-D pharmacophore, which can be used as selection criteria or filter for database screening.

In some preferred embodiments of the present invention, the candidate PARP inhibitor is an analog of 5-iodo-6-nitrocoumarin. Based on the interaction of the 5-iodo-6-nitrocoumarin with the binding domain of the PARP protein, a candidate PARP inhibitor can be designed. Preferably, PARP protein is PARP 1 protein. The candidate PARP inhibitor can include replacement of either iodo or nitro substituent of 5-iodo-6-nitrocoumarin with another substituent. Preferably, the candidate PARP inhibitor can include a replacement of the iodo substituent with another substituent that improves the binding of the candidate PARP inhibitor with the binding domain of the PARP protein.

In some embodiments, the compound is of formula I, its pharmaceutically acceptable salts or prodrugs thereof: 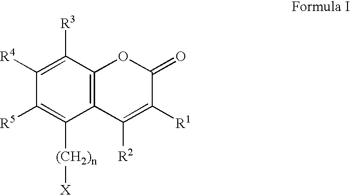
wherein n=0-15; R¹, R², R³, R⁴, R⁵ and X are independently selected from a group consisting of hydrogen, hydroxy, optionally substituted amine, carboxyl, ester, nitroso, nitro, halogen, optionally substituted (C₁-C₆) alkyl, optionally substituted (C₁-C₆) alkoxy, optionally substituted (C₃-C₇) cycloalkyl, optionally substituted (C₃-C₇) heterocyclic, and optionally substituted aryl; and wherein at least two of the R¹, R², R³, R⁴, and R⁵ substituents are always hydrogen. Preferably, n=0-10, or more preferably n=0-5. In some embodiments, n is 0, 1, 2, 3, 4, 5, 6, 7, 8, 9 or 10. In some embodiments, n is 1, 2, 3, 4, 5, 6, 7, 8, 9 or 10. In some embodiments, the halogen is selected from the group consisting of I, Br and Cl. In some embodiments, the halogen is Cl or Br. In some embodiments wherein R⁵ is amino, nitro or nitroso, n is 1, 2, 3, 4, 5, 6, 7, 8, 9 or 10. In some embodiments in which R⁵ is amino, nitro or nitroso, and n is 0, 1, 2, 3, 4, 5, 6, 7, 8, 9 or 10, X is optionally substituted (C₁-C₆) alkyl, optionally substituted (C₁-C₆) alkoxy, optionally substituted (C₃-C₇) cycloalkyl, optionally substituted (C₃-C₇) heterocyclic or optionally substituted aryl. Preferably, the compound is a candidate PARP inhibitor.

In some embodiments of the present invention, the compound is of formula II, its pharmaceutically acceptable salts or prodrugs thereof: 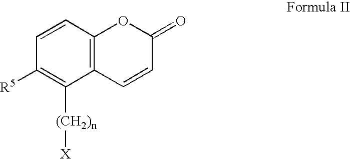
wherein R⁵ is selected from a group consisting of carboxyl, nitroso, and nitro; and X is selected from a group consisting of optionally substituted (C₁-C₇) alkyl, optionally substituted (C₁-C₆) alkoxy, optionally substituted (C₃-C₇) cycloalkyl, optionally substituted (C₃-C₇) heterocyclic, and optionally substituted aryl; and pharmaceutically acceptable salts thereof. Preferably, R⁵ is nitro or nitroso. More preferably, R⁵ is nitro. Preferably, the compound is a candidate PARP inhibitor.

In some embodiments of the present invention, the compound is of formula III, its pharmaceutically acceptable salts or prodrugs thereof: 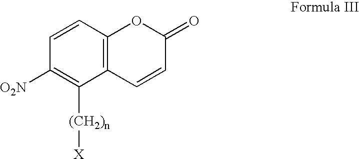
wherein n=0-10, and wherein X is selected from a group consisting of optionally substituted (C₃-C₇) cycloalkyl, optionally substituted (C₃-C₇) heterocyclic, and optionally substituted aryl. Preferably, the compound is a candidate PARP inhibitor.

In some preferred embodiments, the compound is of formula IIIa, its pharmaceutically acceptable salts or prodrugs thereof:

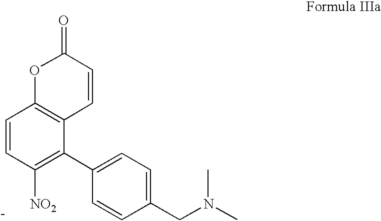

In some preferred embodiments, the compound is of formula IIIb, its pharmaceutically acceptable salts or prodrugs thereof:

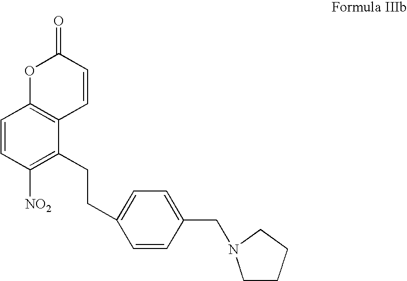

In some embodiments, the optionally substituted (C₃-C₇) heterocyclic is a five membered heterocyclic ring or a six membered heterocyclic ring. In some preferred embodiments, the optionally substituted (C₃-C₇) heterocyclic contains at least one nitrogen. In some embodiments, the optionally substituted (C₃-C₇) heterocyclic is selected from a group consisting of azeridine, azetidine, pyrrole, dihydropyrrole, pyrrolidene, pyrazole, pyrazoline, pyrazolidine, imidazole, benzimidazole, triazole, tetrazole, oxazole, isoxazole, benzoxazole, oxadiazole, oxazoline, oxazolidine, thiazole, isothiazole, pyridine, dihydropyridine, tetrahydropyridine, quinazoline, pyrazine, pyrimidine, pyridazine, quinoline, isoquinoline, triazine, tetrazine, and piperazine.

In some embodiments, the optionally substituted (C₃-C₇) heterocyclic is substituted with a group selected from a group consisting of optionally substituted (C¹-C₆) alkyl, optionally substituted (C₁-C₆) alkoxy, optionally substituted (C₃-C₇) cycloalkyl, optionally substituted (C₃-C₇) heterocyclic, and optionally substituted aryl.

In some preferred embodiments, the compound is of formula IIIc, its pharmaceutically acceptable salts or prodrugs thereof:

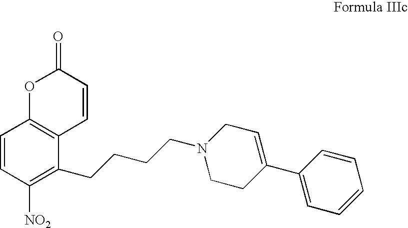

In some preferred embodiments, the compounds is of formula IIId, its pharmaceutically acceptable salts or prodrugs thereof:

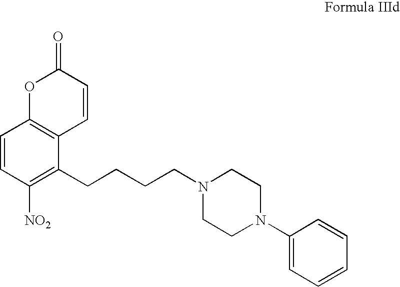

In some preferred embodiments, the compound is of formula IIIe, its pharmaceutically acceptable salts or prodrugs thereof:

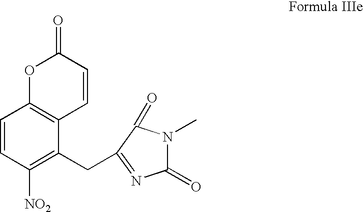

In some preferred embodiments, the compound is of formula IIIf, its pharmaceutically acceptable salts or prodrugs thereof:

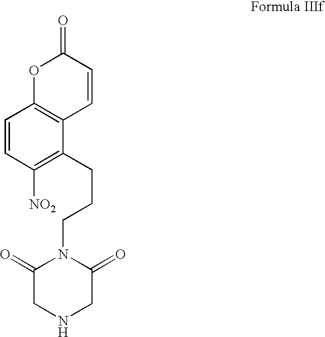

Typical salts are those of the inorganic ions, such as, for example, sodium, potassium, calcium, magnesium ions, and the like. Such salts include salts with inorganic or organic acids, such as hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid or maleic acid. In addition, if the compound(s) contain a carboxy group or other acidic group, it can be converted into a pharmaceutically acceptable addition salt with inorganic or organic bases. Examples of suitable bases include sodium hydroxide, potassium hydroxide, ammonia, cyclohexylamine, dicyclohexyl-amine, ethanolamine, diethanolamine, triethanolamine, and the like.

The PARP inhibitors described herein can contain one or more asymmetric centers and thus occur as racemates and racemic mixtures, single enantiomers, individual diastereomers and diastereomeric mixtures. All such isomeric forms of these compounds are expressly included in the present invention. The PARP inhibitors described herein can also be represented in multiple tautomeric forms, all of which are included herein. The PARP inhibitors can also occur in cis- or trans- or E- or Z-double bond isomeric forms. All such isomeric forms of such inhibitors are expressly included in the present invention. All crystal forms of the PARP inhibitors described herein are expressly included in the present invention. The PARP inhibitors can also be present as their pharmaceutically acceptable salts, derivatives or prodrugs.

Other PARP inhibitors known in the art can also be used as known PARP inhibitors or candidate PARP inhibitors as disclosed in the present invention. The PARP inhibitors have been designed as analogs of benzamides, which bind competitively with the natural substrate NAD in the catalytic site of PARP. The PARP inhibitors include, but are not limited to, benzamides, quinolones and isoquinolones, benzopyrones, methyl 3,5-diiodo-4-(4′-methoxyphenoxy)benzoate, and 3,5-diiodo-4-(4′-methoxyphenoxy)acetophenone (U.S. Pat. No. 5,464,871, U.S. Pat. No. 5,670,518, U.S. Pat. No. 6,004,978, U.S. Pat. No. 6,169,104, U.S. Pat. No. 5,922,775, U.S. Pat. No. 6,017,958, U.S. Pat. No. 5,736,576, and U.S. Pat. No. 5,484,951, all incorporated herein in their entirety). The PARP inhibitors include a variety of cyclic benzamide analogs (i.e. lactams) which are potent inhibitors at the NAD site. Other PARP inhibitors include, but are not limited to, benzimidazoles and indoles (EP841924, EP1127052, U.S. Pat. No. 6,100,283, U.S. Pat. No. 6,310,082, US2002/156050, US2005/054631, WO05/012305, WO99/11628, and US2002/028815). Other PARP inhibitors known in the art can also be used as known PARP inhibitors or candidate PARP inhibitors as disclosed in the present invention (U.S. Application No. 60/804,563, filed on Jun. 12, 2006, incorporated herein by reference in its entirety).

The known or a candidate PARP inhibitor molecule can be examined through the use of computer modeling using a docking program such as GRID, DOCK, or AUTODOCK (see Wolfgang B. Fischer, Anal Bioanal. Chem. 2003, 375, 23-25). This procedure can include computer fitting of a three dimensional structure of a known or a candidate PARP inhibitor molecule to a binding domain of the PARP protein to ascertain how well the shape and the chemical structure of the known or the candidate PARP inhibitor molecule will complement the binding domain of the PARP protein. Computer programs can also be employed to estimate the attraction, repulsion, and steric hindrance of the known or the candidate PARP inhibitor to the binding domain of the PARP protein. Typically, the tighter the fit (e.g., the lower the steric hindrance, and/or the greater the attractive force) the more potent the PARP inhibitor will be since these properties are consistent with a tighter binding constant. Furthermore, the more specificity in the design of a candidate PARP inhibitor the more likely it can be that the candidate PARP inhibitor will not interfere with other properties of the PARP protein or other proteins. This can minimize potential side-effects due to unwanted interactions with other proteins.

Numerous computer programs are available and suitable for a drug design and the processes of computer modeling, model building, and computationally identifying, selecting and evaluating candidate PARP inhibitors in the methods described herein. These include, for example, GRID (available form Oxford University, UK), MCSS (available from Molecular Simulations Inc., Burlington, Mass.), AUTODOCK (available from Oxford Molecular Group), FLEX X (available from Tripos, St. Louis. Mo.), DOCK (available from University of California, San Francisco), CAVEAT (available from University of California, Berkeley), HOOK (available from Molecular Simulations Inc., Burlington, Mass.), and 3D database systems such as MACCS-3D (available from MDL Information Systems, San Leandro, Calif.), UNITY (available from Tripos, St. Louis. Mo.), and CATALYST (available from Molecular Simulations Inc., Burlington, Mass.). The computer program used in the present invention is ICM (available from Molsoft LLC, La Jolla, Calif.).

Potential PARP inhibitors can also be computationally designed “de novo” using such software packages as LUDI (available from Biosym Technologies, San Diego, Calif.), LEGEND (available from Molecular Simulations Inc., Burlington, Mass.), and LEAPFROG (Tripos Associates, St. Louis, Mo.). Compound deformation energy and electrostatic repulsion, can be evaluated using programs such as GAUSSIAN 92, AMBER, QUANTA/CHARMM, AND INSIGHT II/DISCOVER. These computer evaluation and modeling techniques can be performed on any suitable hardware including for example, workstations available from Silicon Graphics, Sun Microsystems, and the like. The computer workstation used in the present invention is Apple Power Mac G5.

The techniques, methods, hardware and software as disclosed herein are representative and are not intended to be limiting to the scope of the present invention. Other modeling techniques known in the art can also be employed in accordance with this invention.

Another aspect of the invention relates to a computer system containing a set of information to perform a design of a PARP inhibitor having a user interface comprising a display unit, the set of information comprising: a) logic for inputting an information regarding a binding of a PARP protein to a known PARP inhibitor; b) logic for designing a candidate PARP inhibitor based on the binding of the PARP protein and known PARP inhibitor; c) logic for determining an information regarding a binding of the PARP protein to the candidate PARP inhibitor; and d) logic for making a conclusion regarding the PARP inhibitory properties of the candidate PARP inhibitor based on the determination of step c).

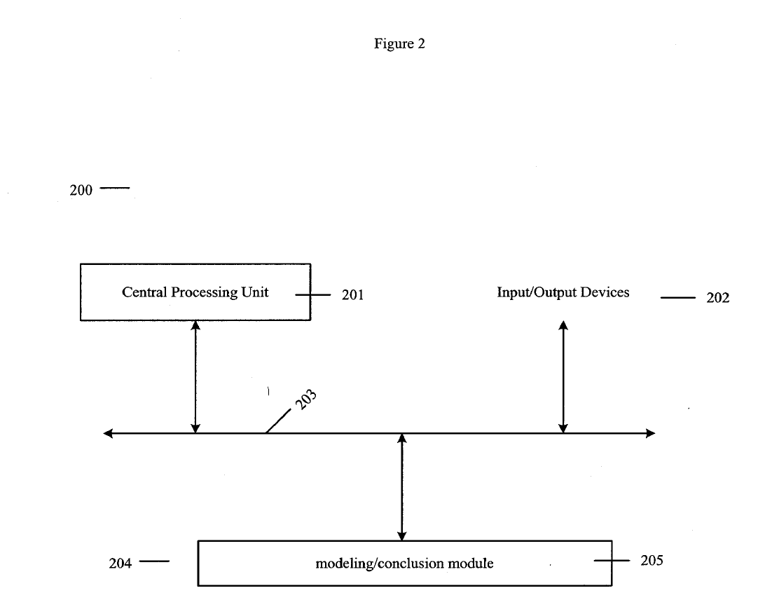

In some preferred embodiments, the steps of the methods of the present invention are performed using a computer as depicted in FIG. 2. FIG. 2 illustrates a computer for implementing selected operations associated with the methods of the present invention. The computer 200 includes a central processing unit 201 connected to a set of input/output devices 202 via a system bus 203. The input/output devices 202 can include a keyboard, mouse, scanner, data port, video monitor, liquid crystal display, printer, and the like. A memory 204 in the form of primary and/or secondary memory is also connected to the system bus 203. These components of FIG. 2 characterize a standard computer. This standard computer is programmed in accordance with the invention. In particular, the computer 200 can be programmed to perform various operations of the methods of the present invention.

The memory 204 of the computer 200 can store a modeling/determining module 205. In other words, the modeling/determining module 205 can perform the operations associated with steps of FIG. 1. The modeling/determining module includes modeling a three dimensional structure of a PARP protein from a crystal of the PARP protein, modeling a three dimensional structure of a binding domain of the PARP protein, modeling a three dimensional structure of a known PARP inhibitor, modeling and determining a binding of the three dimensional structure of the binding domain of the PARP protein with the PARP inhibitor, modeling a three dimensional structure of a candidate PARP inhibitor, modeling and determining a binding of the three dimensional structure of the binding domain of the PARP protein with the candidate PARP inhibitor, and evaluating the binding of the known PARP inhibitor or the candidate PARP inhibitor with the PARP protein. The modeling module can also include a conclusion module which includes a conclusion regarding the candidate PARP inhibitor that inhibits PARP.

The candidate PARP inhibitor as disclosed herein can be prepared by employing standard synthetic techniques known in the art. The candidate PARP inhibitors can be analyzed for their bioactivity. Preferably, the bioactivity relates to inhibition of PARP activity. The compounds which display PARP inhibiting activity can be candidate PARP inhibitors, while the compounds which do not display PARP inhibiting activity help define portions of the molecule which are particularly involved in imparting PARP inhibiting activity to the candidate PARP inhibitor. Where analog compounds are not bioactive, additional analog compounds can be designed, subjected to the methods of the present invention, and then tested for bioactivity. Additional candidate PARP inhibitors can be devised by either repeating the above-described process, or seeking to render other portions of the target structure chemically modified.

In some embodiments of the present invention, pertinent physical and chemical properties (i.e., sites of hydrogen bonding, surface area, atomic and molecular volume, charge density, directionality of the charges, etc.) of candidate PARP inhibitors can be used to develop a collection of parameters required for the desired bioactivity. A database of known compounds (e.g., the Cambridge crystal structure database) can then be searched for structures which contain the steric parameters required for the desired bioactivity. Compounds which are found to contain the desired steric parameters can be retrieved, and further analyzed to determine which of the retrieved compounds also have the desired electronic properties, relative to the candidate PARP inhibitor. Compounds that are found to contain both the desired steric and electronic properties can be additional candidates as PARP inhibitors.

Known compounds which also possess the collection of parameters required for the desired bioactivity can then be tested to see if they also possess the desired bioactivity. Alternatively, known compounds which also possess the collection of parameters required for the desired bioactivity can be modified to remove excess functionality which is not required for the particular bioactivity being tested. Such a modified compound can be a simple, readily prepared PARP inhibitor.

The PARP inhibitors described herein are also useful for inhibiting the biological activity of any enzyme comprising greater than 90%, alternatively greater than 85%, or alternatively greater than 70% sequence homology with a PARP protein sequence. The PARP inhibitors described herein are also useful for inhibiting the biological activity of any enzyme comprising a subsequence, or variant thereof, of any enzyme that comprises greater than 90%, alternatively greater than 85%, or alternatively greater than 70% sequence homology with a PARP protein subsequence. Such subsequence preferably comprises greater than 90%, alternatively greater than 85%, or alternatively greater than 70% sequence homology with the sequence of an active site or subdomain of a PARP protein.

Synthesizing PARP Inhibitors

The candidate PARP inhibitors as disclosed herein can be prepared by employing standard synthetic techniques known in the art and such techniques are within the scope of the present invention. Without limiting the scope of the present invention some of the synthesis schemes for the candidate PARP inhibitors are provided as below.

5-Iodo-6-nitrobenzopyr-2-one (INBP or 5-iodo-6-nitrocoumarin) may be obtained as described in U.S. Pat. No. 5,484,951, which is incorporated herein by reference in its entirety. In the alternative, the INBP may be obtained according to the following reaction scheme:

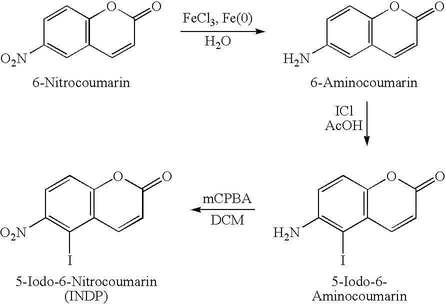

An example of a synthesis scheme for candidate PARP inhibitor of a compound of formula IIIa is as provided below. The (dimethylaminomethyl)phenol (CAS # 25338-55-0) is treated in step (i) with triflic anhydride (see D. Frantz et al., Org. Lett., 2002, 4, p. 4717-4718). Step (ii) forms a borate (H. Nakamura et al. J. Org. Chem. 1998, 63, p. 7529-7530) which reacts with iodonitrocoumarin to give compound of formula IIIa (W. Liu et al. Synthesis, 2006, p 860-864).

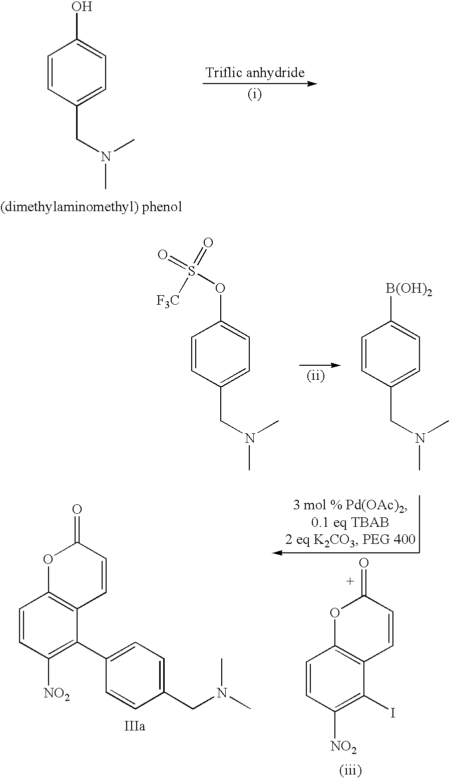

An alternative synthetic scheme for preparing a PARP inhibitor of formula IIIa comprises Suzuki coupling as shown in the following reaction scheme:

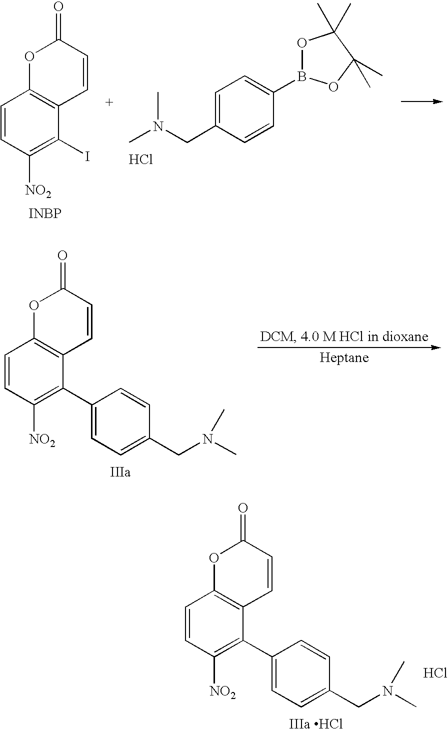

An example of a synthesis scheme for candidate PARP inhibitor of a compound of formula IIIb is as provided below (S. Huo, Org. Lett., 2003, 5, 423-425; T Baughman et al. Tetrahedron, 2004, 60, 10943-10948). Bromoethyl acetate (CAS # 927-68-4) is treated in step (i) with Zn dust to make its corresponding ZnBr, which is then treated with 1(4-iodobenzyl)pyrrolidine (CAS # 858676-60-5) in step (ii). In step (v) 5-iodo-6-nitrocoumarin is treated with a product of step (iv) to give a compound of formula IIIb.

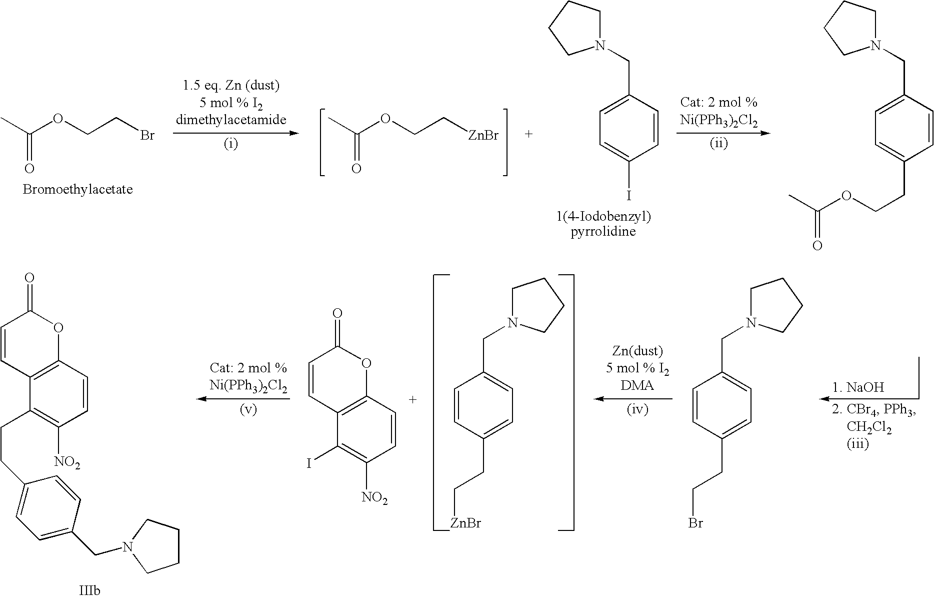

An alternative synthesis scheme for manufacturing IIIb is shown in the following scheme:

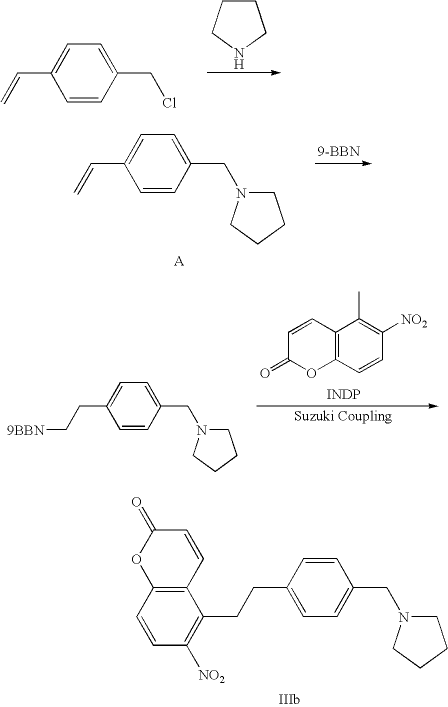

An example of a synthesis scheme for candidate PARP inhibitor of a compound of formula IIIc is as provided below (S. Huo, Org. Lett, 2003, 5, 423-425). 4-Phenyl-1,2,3,6-tetrahydropyridine (CAS #43064-12-6) is treated with 1,4-dibromobutane (CAS # 110-52-1) in step (i). In step (iii) 5-iodo-6-nitrocoumarin is treated with a product of step (ii) to give a compound of formula IIIc.

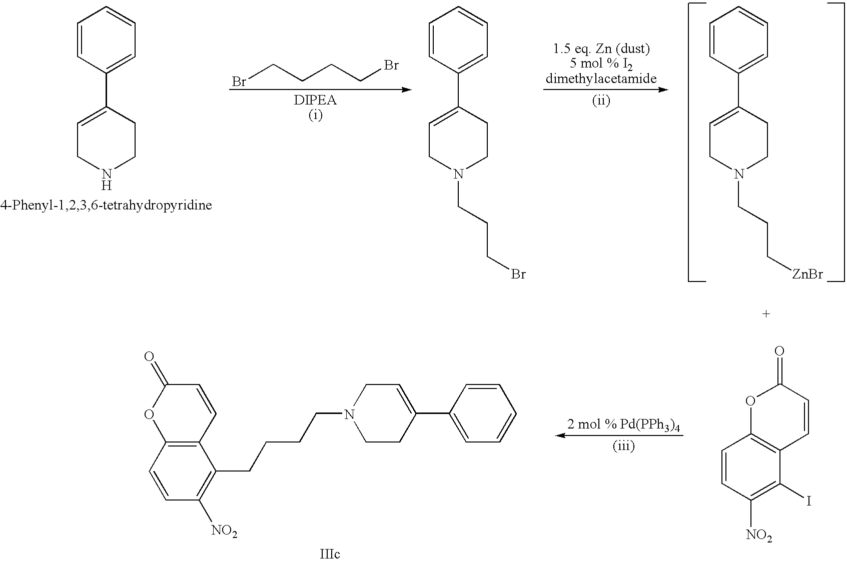

An alternative scheme for synthesizing IIIc is shown in the following scheme:

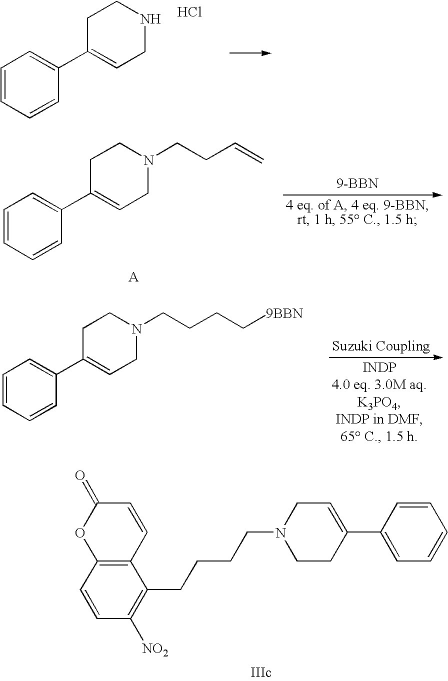

An example of a synthesis scheme for candidate PARP inhibitor of a compound of formula IIId is as provided below (S. Huo, Org. Lett., 2003, 5, 423-425). 1-Phenylpiperazine (CAS # 92-54-6) is treated with 1,4-dibrome butane (CAS # 110-52-1) in step (i). In step (iii) 5-iodo-6-nitrocoumarin is treated with a product of step (ii) to give a compound of formula IIId.

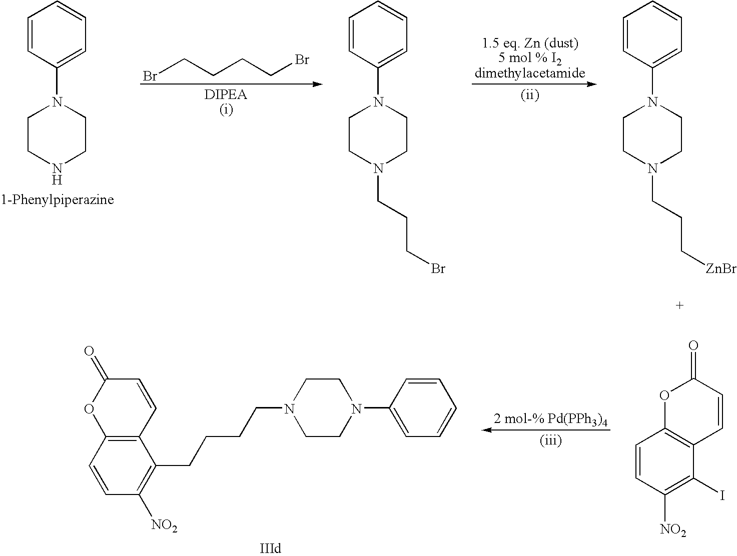

An alternative scheme for preparation of IIId is shown below:

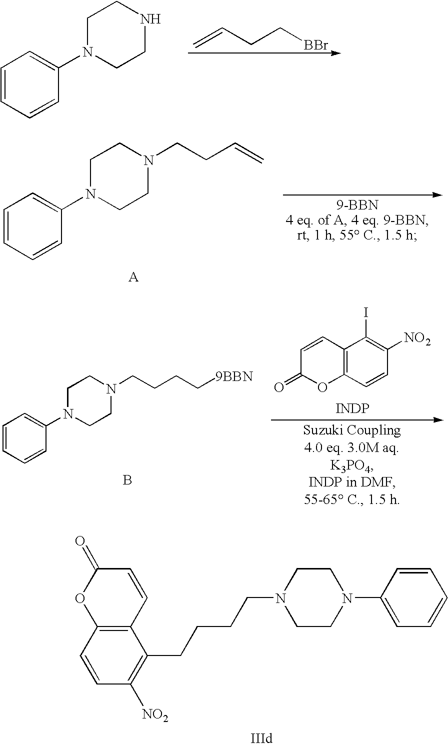

An example of a synthesis scheme for manufacturing IIIe is shown below:

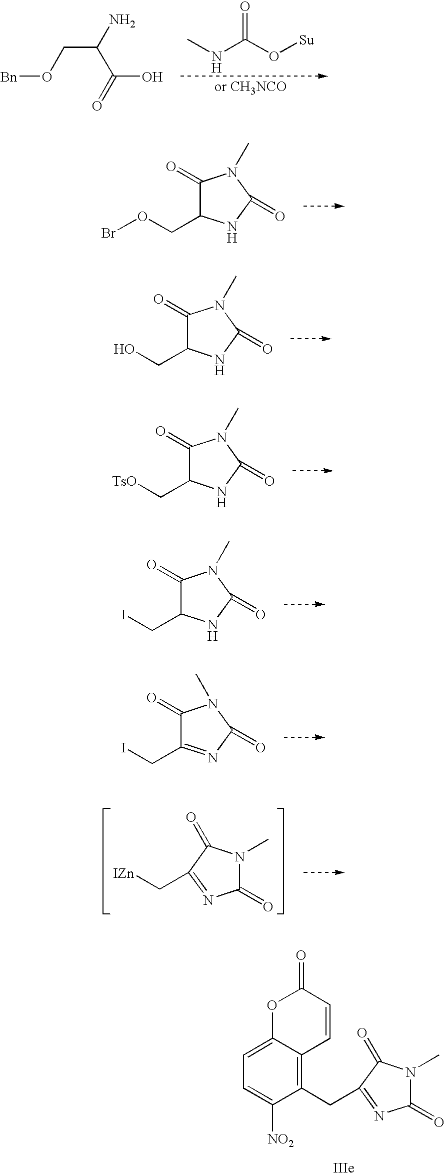

It is suspected, but unconfirmed that IIIe may tautomerize to the enamine form as shown below. This could give rise to E/Z isomers.

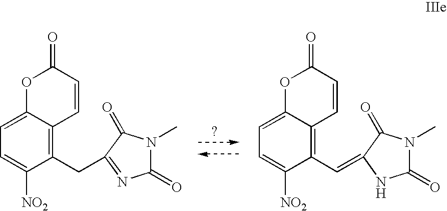

An example of a synthesis scheme for candidate PARP inhibitor of a compound of formula IIIf is as provided below (S. Huo, Org. Lett., 2003, 5, 423-425; T Baughman et al. Tetrahedron, 2004, 60, 10943-10948).

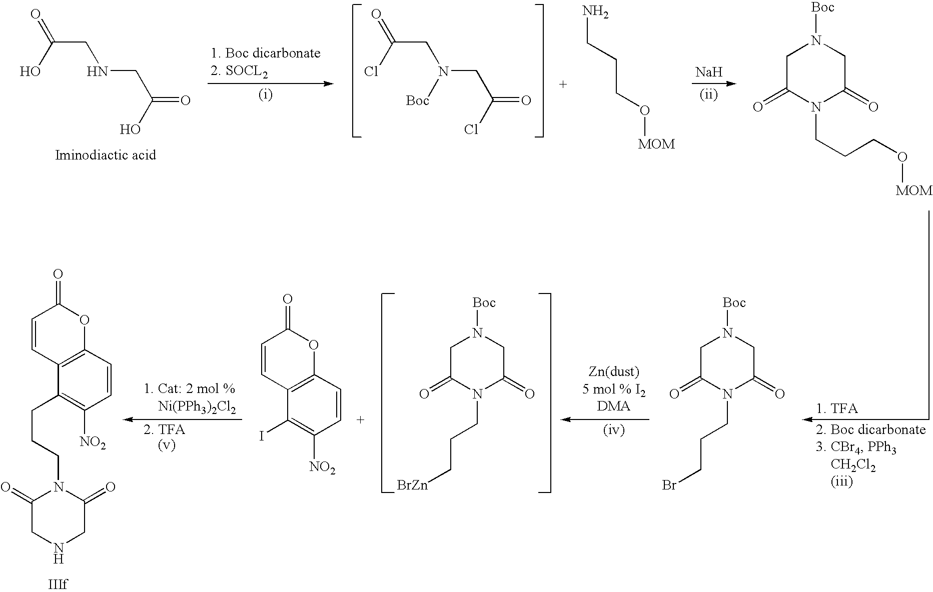

An alternative scheme for synthesis of IIIf is shown below:

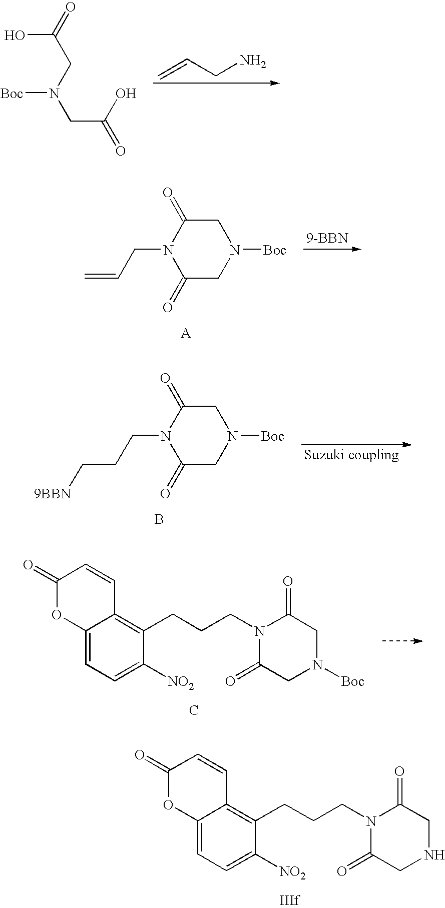

Techniques for Measurement of PARP Inhibiting Activity of PARP Inhibitors

In some embodiments, a PARP inhibiting activity of the candidate PARP inhibitor is evaluated to characterize the ability of a candidate PARP inhibitor to bind to a PARP protein, and/or characterize the ability of the candidate PARP inhibitor to modify the activity of a PARP protein. There are various techniques known in the art to analyze PARP activity. Such techniques include without limitation, mass spectrometry, high performance liquid chromatography etc. Preferably, the technique used for evaluation is an assay technique. Both in vitro and in vivo assays can be used in accordance with the methods of the invention depending on the identity of the PARP protein being investigated. Appropriate activity or functional assays can be readily determined by the skilled artisan based on the disclosure herein. The candidate PARP inhibitors described herein can be used in assays, including radiolabeled, antibody detection and fluorometric assays, for the isolation, identification, or structural or functional characterization of the PARP protein.

The assay can be an enzyme inhibition assay utilizing a full length or truncated PARP protein. The PARP protein can be contacted with the candidate PARP inhibitor and a measurement of the binding affinity of the candidate PARP inhibitor against a standard is determined. Such assays are known to one of ordinary skill in the art and are within the scope of the present invention. The assay for evaluating PARP inhibiting activity of the candidate PARP inhibitor can be a cell-based assay. The candidate PARP inhibitor is contacted with a cell and a measurement of an inhibition of a standard marker produced in the cell is determined. Cells can be either isolated from an animal, including a transformed cultured cell, or can be in a living animal. Such assays are also known to one of ordinary skill in the art and are within the scope of the present invention.

An example of an assay for measuring PARP activity can proceed as follows. PARP-1 is purified from calf thymus as reported earlier (Molinet et al. (1993) EMBO J. 12:2109-2117). Alternatively recombinant PARP-1 is isolated from Sodoptera Fugiperda (Sf9) cells infected with recombinant baculovirus, expressing the human PARP-1 gene, constructed according to the instructions of Pharmingen. The cDNA of the amino acid exchange mutant R34G and R138 il of PARP-1 is created by the mega primer method (Kannann et al. (1989) Nucl Acids Res 17:5404). The mutated gene is cloned into the transfer vector pV 1392 and the recombinant virus is generated by the Baculogold technology of Pharmigen. The mutated proteins are expressed in Sf9 cells, purified and assayed as reported (Huang et al. (2004) Biochemistry 43:217-223; Kirsten et al. (2004) Methods in Molecular Biology 287, Epigenetics Protocols 137-149). Assays can be carried out as described in Kun et al. (2004) Biochemistry, 43:210-216.

The candidate PARP inhibitors of the present invention can be identified using, for example, immunoassays such as enzyme linked immunoabsorbent assays (ELISA) and radioimmunoassays (RIA) or binding assays such as Biacore assays. Binding assays can employ kinetic or thermodynamic methodology using a wide variety of techniques including, but not limited to, microcalorimetry, circular dichroism, capillary zone electrophoresis, nuclear magnetic resonance spectroscopy, fluorescence spectroscopy, and combinations thereof. Without limiting the scope of the present invention, some of the examples of the techniques for measurement of the bioactivity of the PARP inhibitors, are provided below.

Fluorescence Microscopy: Some embodiments of the invention include fluorescence microscopy for measuring the PARP inhibiting activity of the candidate PARP inhibitors of the present invention. Fluorescence microscopy enables the molecular composition of the structures being observed to be identified through the use of fluorescently-labeled probes of high chemical specificity such as antibodies. It can be done by directly conjugating a fluorophore to a PARP protein and introducing this back into a cell. Fluorescent analogue can behave like the native protein and can therefore serve to reveal the distribution and behavior of this PARP protein in the cell. Along with NMR, infrared spectroscopy, circular dichroism and other techniques, protein intrinsic fluorescence decay and its associated observation of fluorescence anisotropy, collisional quenching and resonance energy transfer are techniques for PARP detection. The naturally fluorescent proteins can be used as fluorescent probes. The jellyfish aequorea victoria produces a naturally fluorescent protein known as green fluorescent protein (GFP). The fusion of these fluorescent probes to a target protein enables visualization by fluorescence microscopy and quantification by flow cytometry.

By way of example only, some of the probes are labels such as, fluorescein and its derivatives, carboxyfluoresceins, rhodamines and their derivatives, atto labels, fluorescent red and fluorescent orange: cy3/cy5 alternatives, lanthanide complexes with long lifetimes, long wavelength labels—up to 800 mm, DY cyanine labels, and phycobili proteins. By way of example only, some of the probes are conjugates such as, isothiocyanate conjugates, streptavidin conjugates, and biotin conjugates. By way of example only, some of the probes are enzyme substrates such as, fluorogenic and chromogenic substrates. By way of example only, some of the probes are fluorochromes such as, FITC (green fluorescence, excitation/emission=506/529 nm), rhodamine B (orange fluorescence, excitation/emission=560/584 nm), and Nile blue A (red fluorescence, excitation/emission=636/686 nm). Fluorescent nanoparticles can be used for various types of immunoassays. Fluorescent nanoparticles are based on different materials, such as, polyacrylonitrile, and polystyrene etc. Fluorescent molecular rotors are sensors of microenvironmental restriction that become fluorescent when their rotation is constrained. Few examples of molecular constraint include increased dye (aggregation), binding to antibodies, or being trapped in the polymerization of actin. IEF (isoelectric focusing) is an analytical tool for the separation of ampholytes, mainly proteins. An advantage for IEF-gel electrophoresis with fluorescent IEF-marker is the possibility to directly observe the formation of gradient. Fluorescent IEF-marker can also be detected by UV-absorption at 280 nm (20° C.).

A peptide library can be synthesized on solid supports and, by using coloring receptors, subsequent dyed solid supports can be selected one by one. If receptors cannot indicate any color, their binding antibodies can be dyed. The method can not only be used on protein receptors, but also on screening binding ligands of synthesized artificial receptors and screening new metal binding ligands as well. Automated methods for HTS and FACS (fluorescence activated cell sorter) can also be used.

Immunoassays: Some embodiments of the invention include immunoassay for measuring the PARP inhibiting activity of the candidate PARP inhibitors of the present invention. In immunoblotting like the western blot of electrophoretically separated proteins a single protein can be identified by its antibody. Immunoassay can be competitive binding immunoassay where analyte competes with a labeled antigen for a limited pool of antibody molecules (e.g. radioimmunoassay, EMIT). Immunoassay can be non-competitive where antibody is present in excess and is labeled. As analyte antigen complex is increased, the amount of labeled antibody-antigen complex can also increase (e.g. ELISA). Antibodies can be polyclonal if produced by antigen injection into an experimental animal, or monoclonal if produced by cell fusion and cell culture techniques. In immunoassay, the antibody can serve as a specific reagent for the analyte antigen.

Without limiting the scope and content of the present invention, some of the types of immunoassays are, but not limited to, RIAs (radioimmunoassay), enzyme immunoassays like ELISA (enzyme-linked immunosorbent assay), EMIT (enzyme multiplied immunoassay technique), microparticle enzyme immunoassay (MEIA), LIA (luminescent immunoassay), and FIA (fluorescent immunoassay). The antibodies—either used as primary or secondary ones—can be labeled with radioisotopes (e.g. 125I), fluorescent dyes (e.g. FITC) or enzymes (e.g. HRP or AP) which can catalyze fluorogenic or luminogenic reactions.

Biotin, or vitamin H is a co-enzyme which inherits a specific affinity towards avidin and streptavidin. This interaction makes biotinylated peptides a useful tool in various biotechnology assays for quality and quantity testing. To improve biotin/streptavidin recognition by minimizing steric hindrances, it can be necessary to enlarge the distance between biotin and the peptide itself. This can be achieved by coupling a spacer molecule (e.g., 6-aminohexanoic acid) between biotin and the peptide.

The biotin quantitation assay for biotinylated proteins provides a sensitive fluorometric assay for accurately determining the number of biotin labels on a protein. Biotinylated peptides are widely used in a variety of biomedical screening systems requiring immobilization of at least one of the interaction partners onto streptavidin coated beads, membranes, glass slides or microtiter plates. The assay is based on the displacement of a ligand tagged with a quencher dye from the biotin binding sites of a reagent. To expose any biotin groups in a multiply labeled protein that are sterically restricted and inaccessible to the reagent, the protein can be treated with protease for digesting the protein.

EMIT is a competitive binding immunoassay that avoids the usual separation step. A type of immunoassay in which the protein is labeled with an enzyme, and the enzyme-protein-antibody complex is enzymatically inactive, allowing quantitation of unlabelled protein. Some embodiments of the invention include ELISA to analyze PARP. ELISA is based on selective antibodies attached to solid supports combined with enzyme reactions to produce systems capable of detecting low levels of proteins. It is also known as enzyme immunoassay or EIA. The protein is detected by antibodies that have been made against it, that is, for which it is the antigen. Monoclonal antibodies are often used.

The test can require the antibodies to be fixed to a solid surface, such as the inner surface of a test tube, and a preparation of the same antibodies coupled to an enzyme. The enzyme can be one (e.g., β-galactosidase) that produces a colored product from a colorless substrate. The test, for example, can be performed by filling the tube with the antigen solution (e.g., protein) to be assayed. Any antigen molecule present can bind to the immobilized antibody molecules. The antibody-enzyme conjugate can be added to the reaction mixture. The antibody part of the conjugate binds to any antigen molecules that were bound previously, creating an antibody-antigen-antibody “sandwich”. After washing away any unbound conjugate, the substrate solution can be added. After a set interval, the reaction is stopped (e.g., by adding 1 N NaOH) and the concentration of colored product formed is measured in a spectrophotometer. The intensity of color is proportional to the concentration of bound antigen.

ELISA can also be adapted to measure the concentration of antibodies, in which case, the wells are coated with the appropriate antigen. The solution (e.g., serum) containing antibody can be added. After it has had time to bind to the immobilized antigen, an enzyme-conjugated anti-immunoglobulin can be added, consisting of an antibody against the antibodies being tested for. After washing away unreacted reagent, the substrate can be added. The intensity of the color produced is proportional to the amount of enzyme-labeled antibodies bound (and thus to the concentration of the antibodies being assayed).

Some embodiments of the invention include radioimmunoassays for measuring the PARP inhibiting activity of the candidate PARP inhibitors of the present invention. Radioactive isotopes can be used to study in vivo metabolism, distribution, and binding of small amount of compounds. Radioactive isotopes of ¹H, ¹²C, ³¹P, ³²S, and ¹²⁷I in body are used such as ³H, ¹⁴C, ³²P, ³⁵S, and ¹²⁵I. In receptor fixation method in 96 well plates, receptors can be fixed in each well by using antibody or chemical methods and radioactive labeled ligands can be added to each well to induce binding. Unbound ligands can be washed out and then the standard can be determined by quantitative analysis of radioactivity of bound ligands or that of washed-out ligands. Then, addition of screening target compounds can induce competitive binding reaction with receptors. If the compounds show higher affinity to receptors than standard radioactive ligands, most of radioactive ligands would not bind to receptors and can be left in solution. Therefore, by analyzing quantity of bound radioactive ligands (or washed-out ligands), testing compounds' affinity to receptors can be indicated.

The filter membrane method can be needed when receptors cannot be fixed to 96 well plates or when ligand binding needs to be done in solution phase. In other words, after ligand-receptor binding reaction in solution, if the reaction solution is filtered through nitrocellulose filter paper, small molecules including ligands can go through it and only protein receptors can be left on the paper. Only ligands that strongly bound to receptors can stay on the filter paper and the relative affinity of added compounds can be identified by quantitative analysis of the standard radioactive ligands.

Some embodiments of the invention include fluorescence immunoassays for measuring the PARP inhibiting activity of the candidate PARP inhibitors of the present invention. Fluorescence based immunological methods are based upon the competitive binding of labeled ligands versus unlabeled ones on highly specific receptor sites. The fluorescence technique can be used for immunoassays based on changes in fluorescence lifetime with changing analyte concentration. This technique can work with short lifetime dyes like fluorescein isothiocyanate (FITC) (the donor) whose fluorescence can be quenched by energy transfer to eosin (the acceptor). A number of photoluminescent compounds can be used, such as cyanines, oxazines, thiazines, porphyrins, phthalocyanines, fluorescent infrared-emitting polynuclear aromatic hydrocarbons, phycobiliproteins, squaraines and organo-metallic complexes, hydrocarbons and azo dyes.

Fluorescence based immunological methods can be, for example, heterogeneous or homogenous. Heterogeneous immunoassays comprise physical separation of bound from free labeled analyte. The analyte or antibody can be attached to a solid surface. Homogenous immunoassays comprise no physical separation. Double-antibody fluorophore-labeled antigen participates in an equilibrium reaction with antibodies directed against both the antigen and the fluorophore. Labeled and unlabeled antigen can compete for a limited number of anti-antigen antibodies.

Some of the fluorescence immunoassay methods include simple fluorescence labeling method, fluorescence resonance energy transfer (FRET), time resolved fluorescence (TRF), and scanning probe microscopy (SPM). The simple fluorescence labeling method can be used for receptor-ligand binding, enzymatic activity by using pertinent fluorescence, and as a fluorescent indicator of various in vivo physiological changes such as pH, ion concentration, and electric pressure.

Method of Treatment with PARP Inhibitors

The present invention relates to a pharmaceutical composition, medicament, drug or other composition of the candidate PARP inhibitors comprising compounds of formula I-III where III includes IIIa-f, for treatment of diseases. Preferably, the diseases are PARP mediated diseases. The candidate PARP inhibitors of the present invention can have therapeutic benefit in the treatment of various diseases such as, cardiovascular disease, cancer, metabolic disease, myocardial ischemia, stroke, head trauma, neurodegenerative disease, and as an adjunct therapy with chemotherapeutic agents/radiation in cancer therapy.

The methods of the present invention also comprise the administration of candidate PARP inhibitors in combination with other therapies. The choice of therapy that can be co-administered with the compositions of the invention will depend, in part, on the condition being treated. For example, for treating cancer, compound of some embodiments of the invention can be used in combination with radiation therapy, monoclonal antibody therapy, chemotherapy, bone marrow transplantation, or a combination thereof.

The candidate PARP inhibitors of the present invention can be useful in treating or preventing a variety of diseases and illnesses including neural tissue damage resulting from cell damage or death due to necrosis or apoptosis, cerebral ischemia and reperfusion injury or neurodegenerative diseases in an animal. In addition, the candidate PARP inhibitors of the present invention can also be used to treat a cardiovascular disorder selected from the group consisting of: coronary artery disease, such as atherosclerosis; angina pectoris; myocardial infarction; myocardial ischemia and cardiac arrest; cardiac bypass; and cardiogenic shock. Further still, the compounds of the invention can be used to treat cancer and to radiosensitize or chemosensitize tumor cells.

In another aspect, the candidate PARP inhibitors in the present invention can be used to treat cancer, and to radiosensitize and/or chemosensitize tumor cells. The candidate PARP inhibitors of the present invention can be “anti-cancer agents,” which term also encompasses “anti-tumor cell growth agents” and “anti-neoplastic agents.” Radiosensitizers are known to increase the sensitivity of cancerous cells to the toxic effects of electromagnetic radiation. Many cancer treatment protocols currently employ radiosensitizers activated by the electromagnetic radiation of x-rays. Examples of x-ray activated radiosensitizers include, but are not limited to, the following: metronidazole, misonidazole, desmethylmisonidazole, pimonidazole, etanidazole, nimorazole, mitomycin C, RSU 1069, SR 4233, EO9, RB 6145, nicotinamide, 5-bromodeoxyuridine (BUdR), 5-iododeoxyuridine (IUdR), bromodeoxycytidine, fluorodeoxyuridine (FudR), hydroxyurea, cisplatin, and therapeutically effective analogs and derivatives of the same.

Photodynamic therapy (PDT) of cancers employs visible light as the radiation activator of the sensitizing agent. Examples of photodynamic radiosensitizers include the following, but are not limited to: hematoporphyrin derivatives, photofrin, benzoporphyrin derivatives, NPe6, tin etioporphyrin SnET2, pheoborbide-α, bacteriochlorophyll-α, naphthalocyanines, phthalocyanines, zinc phthalocyanine, and therapeutically effective analogs and derivatives of the same.

Chemosensitizers are also known to increase the sensitivity of cancerous cells to the toxic effects of chemotherapeutic compounds. Exemplary chemotherapeutic agents that can be used in conjunction with PARP inhibitors include, but are not limited to, adriamycin, camptothecin, dacarbazine, carboplatin, cisplatin, daunorubicin, docetaxel, doxorubicin, interferon (alpha, beta, gamma), interleukin 2, innotecan, paclitaxel, streptozotocin, temozolomide, topotecan, and therapeutically effective analogs and derivatives of the same. In addition, other therapeutic agents which can be used in conjunction with a PARP inhibitors include, but are not limited to, 5-fluorouracil, leucovorin, 5′-amino-5′-deoxythymidine, oxygen, carbogen, red cell transfusions, perfluorocarbons (e.g., Fluosol-DA), 2,3-DPG, BW12C, calcium channel blockers, pentoxyfylline, antiangiogenesis compounds, hydralazine, and L-BSO.

The methods of treatment as disclosed herein can be via oral administration, transmucosal administration, buccal administration, nasal administration, inhalation, parental administration, intravenous, subcutaneous, intramuscular sublingual, transdermal administration, and rectal administration.

Pharmaceutical compositions of the candidate PARP inhibitors of the present invention, include compositions wherein the active ingredient is contained in a therapeutically or prophylactically effective amount, i.e., in an amount effective to achieve therapeutic or prophylactic benefit. The actual amount effective for a particular application will depend, inter alia, on the condition being treated and the route of administration. Determination of an effective amount is well within the capabilities of those skilled in the art. The pharmaceutical compositions comprise the candidate PARP inhibitor, one or more pharmaceutically acceptable carriers, diluents or excipients, and optionally additional therapeutic agents. The compositions can be formulated for sustained or delayed release.

A preferred therapeutic composition of the present invention also includes an excipient, an adjuvant and/or carrier. Suitable excipients include compounds that the subject to be treated can tolerate. Examples of such excipients include water, saline, Ringer's solution, dextrose solution, Hank's solution, and other aqueous physiologically balanced salt solutions. Nonaqueous vehicles, such as fixed oils, sesame oil, ethyl oleate, or triglycerides can also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosal, o-cresol, formalin and benzyl alcohol. Standard formulations can either be liquid injectables or solids which can be taken up in a suitable liquid as a suspension or solution for injection. Thus, in a non-liquid formulation, the excipient can comprise dextrose, human serum albumin, preservatives, etc., to which sterile water or saline can be added prior to administration. In one embodiment of the present invention, a therapeutic composition can include a carrier. Carriers include compounds that increase the half-life of a therapeutic composition in the treated subject. Suitable carriers include, but are not limited to, polymeric controlled release vehicles, biodegradable implants, liposomes, bacteria, viruses, other cells, oils, esters, and glycols.

The oral form in which the therapeutic agent is administered can include powder, tablet, capsule, solution, or emulsion. The effective amount can be administered in a single dose or in a series of doses separated by appropriate time intervals, such as hours. Pharmaceutical compositions can be formulated in conventional manner using one or more physiologically acceptable carriers comprising excipients and auxiliaries which facilitate processing of the active compounds into preparations which can be used pharmaceutically. Proper formulation is dependent upon the route of administration chosen. Suitable techniques for preparing pharmaceutical compositions of the therapeutic agents of the present invention are well known in the art.

It will be appreciated that appropriate dosages of the active compounds, and compositions comprising the active compounds, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular candidate PARP inhibitor, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g. in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

Some of the examples of diseases treatable by PARP inhibitors of the present invention are disclosed herein but they are not in any way limiting to the scope of the present invention.

Examples of Various Diseases

Various diseases that can be treated by the candidate PARP inhibitors of the present invention include, but are not limited to, cancers, inflammation, degenerative diseases, CNS diseases, autoimmune diseases, and viral diseases, including HIV. The compounds described herein are also useful in the modulation of cellular response to pathogens. The invention also provides methods to treat other diseases, such as, viral diseases. Some of the viral diseases are, but not limited to, human immunodeficiency virus (HIV), herpes simplex virus type-1 and 2 and cytomegalovirus (CMV), a dangerous co-infection of HIV.

Examples of Cancer

The cancer include but are not limited to, adrenal cortical cancer, anal cancer, aplastic anemia, bile duct cancer, bladder cancer, bone cancer, bone metastasis, Adult CNS brain tumors, Children CNS brain tumors, breast cancer, Castleman Disease, cervical cancer, Childhood Non-Hodgkin's lymphoma, colon and rectum cancer, endometrial cancer, esophagus cancer, Ewing's family of tumors, eye cancer, gallbladder cancer, gastrointestinal carcinoid tumors, gastrointestinal stromal tumors, gestational trophoblastic disease, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, nonmelanoma skin cancer, stomach cancer, testicular cancer, thymus cancer, thyroid cancer, uterine sarcoma, vaginal cancer, vulvar cancer, and Waldenstrom's macroglobulinemia.

Carcinoma of the thyroid gland is the most common malignancy of the endocrine system. Carcinoma of the thyroid gland include differentiated tumors (papillary or follicular) and poorly differentiated tumors (medullary or anaplastic). Carcinomas of the vagina include squamous cell carcinoma, adenocarcinoma, melanoma and sarcoma. Testicular cancer is broadly divided into seminoma and nonseminoma types.

Thymomas are epithelial tumors of the thymus, which may or may not be extensively infiltrated by nonneoplastic lymphocytes. The term thymoma is customarily used to describe neoplasms that show no overt atypia of the epithelial component. A thymic epithelial tumor that exhibits clear-cut cytologic atypia and histologic features no longer specific to the thymus is known as a thymic carcinoma (also known as type C thymoma).

The methods provided by the invention can comprise the administration of the PARP inhibitors in combination with other therapies. The choice of therapy that can be co-administered with the compositions of the invention can depend, in part, on the condition being treated. For example, for treating acute myleoid leukemia, a PARP inhibitor can be used in combination with radiation therapy, monoclonal antibody therapy, chemotherapy, bone marrow transplantation, gene therapy, immunotherapy, or a combination thereof.

Her-2 Related Cancer

Her-2 disease is a type of breast cancer. Characterized by aggressive growth and a poor prognosis, it can be caused by the presence of excessive numbers of a gene called HER2 (human epidermal growth factor receptor-2) in tumor cells. Therapies that can used in combination with the PARP inhibitors as disclosed herein include, but are no limited to Her-2 antibodies such as herceptin, anti-hormones (e.g., selective oestrogen receptor modulator (SERM) tamoxifen), chemotherapy and radiotherapy, aromatase inhibitors (e.g. anastrazole, letrozole and exemestane) and anti-estrogens (e.g., fulvestrant (Faslodex)).

Breast Cancer

A lobular carcinoma in situ and a ductal carcinoma in situ are breast cancers that develop in the lobules and ducts, respectively, but may not have spread to the fatty tissue surrounding the breast or to other areas of the body. An infiltrating (or invasive) lobular and a ductal carcinoma are cancers that have developed in the lobules and ducts, respectively, and have spread to either the breast's fatty tissue and/or other parts of the body. Other cancers of the breast that can benefit from treatment provided by the methods of the present invention are medullary carcinomas, colloid carcinomas, tubular carcinomas, and inflammatory breast cancer.

In some embodiments, the invention provides for treatment of so-called “triple negative” breast cancer. There are several subclasses of breast cancer identified by classic biomarkers such as estrogen receptor (ER) and/or progesterone receptor (PR) positive tumors, HER2-amplified tumors, and ER/PR/HER2-negative tumors. These three subtypes have been reproducibly identified by gene expression profiling in multiple breast cancer and exhibit basal-like subtype expression profiles and poor prognosis. Triple negative breast cancer is characterized by ER/PR/HER2-negative tumors.

Ovarian Cancer

The ovarian cancer includes but is not limited to, epithelial ovarian tumors, adenocarcinoma in the ovary and an adenocarcinoma that has migrated from the ovary into the abdominal cavity. Treatments for ovarian cancer that can be used in combination with the PARP inhibitors of the present invention include but are not limited to, surgery, immunotherapy, chemotherapy, hormone therapy, radiation therapy, or a combination thereof. Some possible surgical procedures include debulking, and a unilateral or bilateral oophorectomy and/or a unilateral or bilateral salpigectomy.

Anti-cancer drugs that can be used in the combination therapy include cyclophosphamide, etoposide, altretamine, and ifosfamide. Hormone therapy with the drug tamoxifen can be used to shrink ovarian tumors. Radiation therapy can be external beam radiation therapy and/or brachytherapy.

Cervical Cancer

The cervical cancer includes, but is not limited to, an adenocarcinoma in the cervix epithelial. Two main types of this cancer exist: squamous cell carcinoma and adenocarcinomas. Some cervical cancers have characteristics of both of these and are called adenosquamous carcinomas or mixed carcinomas.

Prostate Cancer

The prostate cancer includes, but is not limited to, an adenocarcinoma or an adenocarinoma that has migrated to the bone. Prostate cancer develops in the prostate organ in men, which surrounds the first part of the urethra.

Pancreatic Cancer

The pancreatic cancer includes, but is not limited to, an epitheliod carcinoma in the pancreatic duct tissue and an adenocarcinoma in a pancreatic duct. Treatments that can be used in combination with the PARP inhibitors of the present invention include but are not limited to, surgery, immunotherapy, radiation therapy, and chemotherapy. Possible surgical treatment options include a distal or total pancreatectomy and a pancreaticoduodenectomy (Whipple procedure). Radiation therapy can be an option for pancreatic cancer patients, such as external beam radiation where radiation is focused on the tumor by a machine outside the body. Another option is intraoperative electron beam radiation administered during an operation.

Bladder Cancer

The bladder cancer includes, but is not limited to, a transitional cell carcinoma in urinary bladder. Bladder cancers are urothelial carcinomas (transitional cell carcinomas) or tumors in the urothelial cells that line the bladder. The remaining cases of bladder cancer are squamous cell carcinomas, adenocarcinomas, and small cell cancers. Several subtypes of urothelial carcinomas exist depending on whether they are noninvasive or invasive and whether they are papillary, or flat. Noninvasive tumors are in the urothelium, the innermost layer of the bladder, while invasive tumors have spread from the urothelium to deeper layers of the bladder's main muscle wall. Invasive papillary urothelial carcinomas are slender finger-like projections that branch into the hollow center of the bladder and also grow outward into the bladder wall. Non-invasive papillary urothelial tumors grow towards the center of the bladder. While a non-invasive, flat urothelial tumor (also called a flat carcinoma in situ) is confined to the layer of cells closest to the inside hollow part of the bladder, an invasive flat urothelial carcinoma invades the deeper layer of the bladder, particularly the muscle layer.

The therapies that can be used in combination with the PARP inhibitors of the present invention for the treatment of bladder cancer include surgery, radiation therapy, immunotherapy, chemotherapy, or a combination thereof. Some surgical options are a transurethral resection, a cystectomy, or a radical cystectomy. Radiation therapy for bladder cancer can include external beam radiation and brachytherapy.

Immunotherapy is another method that can be used to treat a bladder cancer patient. One method is Bacillus Calmete-Guerin (BCG) where a bacterium sometimes used in tuberculosis vaccination is given directly to the bladder through a catheter. The body mounts an immune response to the bacterium, thereby attacking and killing the cancer cells. Another method of immunotherapy is the administration of interferons, glycoproteins that modulate the immune response. Interferon alpha is often used to treat bladder cancer.

Anti-cancer drugs that can be used in combination to treat bladder cancer include thitepa, methotrexate, vinblastine, doxorubicin, cyclophosphamide, paclitaxel, carboplatin, cisplatin, ifosfamide, gemcitabine, or combinations thereof.

Blood Cancer

Lymphoma

B-Cell Lymphomas

Non-Hodgkin's Lymphomas caused by malignant (cancerous) B-Cell lymphocytes represent a large subset (about 85% in the US) of the known types of lymphoma (the other 2 subsets being T-Cell lymphomas and lymphomas where the cell type is the Natural Killer Cell or unknown). Cells undergo many changes in their life cycle dependent on complex signaling processes between cells and interaction with foreign substances in the body. Various types of lymphoma or leukemia can occur in the B-Cell life cycle.

Acute Myeloid Leukemia

The acute myeloid leukemia (AML) includes acute promyleocytic leukemia in peripheral blood. AML begins in the bone marrow but can spread to other parts of the body including the lymph nodes, liver, spleen, central nervous system, and testes. AML can be characterized by immature bone marrow cells usually granulocytes or monocytes, which can continue to reproduce and accumulate.

AML can be treated by other therapies in combination with the PARP inhibitors of the present invention. Such therapies include but are not limited to, immunotherapy, radiation therapy, chemotherapy, bone marrow or peripheral blood stem cell transplantation, or a combination thereof. Radiation therapy includes external beam radiation and can have side effects. Anti-cancer drugs that can be used in chemotherapy to treat AML include cytarabine, anthracycline, anthracenedione, idarubicin, daunorubicin, idarubicin, mitoxantrone, thioguanine, vincristine, prednisone, etoposide, or a combination thereof.

Monoclonal antibody therapy can be used to treat AML patients. Small molecules or radioactive chemicals can be attached to these antibodies before administration to a patient in order to provide a means of killing leukemia cells in the body. The monoclonal antibody, gemtuzumab ozogamicin, which binds CD33 on AML cells, can be used to treat AML patients unable to tolerate prior chemotherapy regimens. Bone marrow or peripheral blood stem cell transplantation can be used to treat AML patients. Some possible transplantation procedures are an allogenic or an autologous transplant.

Other types of leukemia's that can be treated by the methods provided by the invention include but not limited to, Acute Lymphocytic Leukemia, Chronic Lymphocytic Leukemia, Chronic Myeloid Leukemia, Hairy Cell Leukemia, Myelodysplasia, and Myeloproliferative Disorders.

Lung Cancer

The common type of lung cancer is non-small cell lung cancer (NSCLC), which is divided into squamous cell carcinomas, adenocarcinomas, and large cell undifferentiated carcinomas. Treatment options for lung cancer in combination with the PARP inhibitors of the present invention include surgery, immunotherapy, radiation therapy, chemotherapy, photodynamic therapy, or a combination thereof. Some possible surgical options for treatment of lung cancer are a segmental or wedge resection, a lobectomy, or a pneumonectomy. Radiation therapy can be external beam radiation therapy or brachytherapy.

Some anti-cancer drugs that can be used in chemotherapy to treat lung cancer include cisplatin, carboplatin, paclitaxel, docetaxel, gemcitabine, vinorelbine, irinotecan, etoposde, vinblastine, gefitinib, ifosfamide, methotrexate, or a combination thereof. Photodynamic therapy (PDT) can be used to treat lung cancer patients.

Skin Cancer

There are several types of cancer that start in the skin. The most common types are basal cell carcinoma and squamous cell carcinoma, which are non-melanoma skin cancers. Actinic keratosis is a skin condition that sometimes develops into squamous cell carcinoma. Non-melanoma skin cancers rarely spread to other parts of the body. Melanoma, the rarest form of skin cancer, is more likely to invade nearby tissues and spread to other parts of the body.

Different types of treatments that can be used in combination with the PARP inhibitors of the present invention include but are not limited to, surgery, radiation therapy, chemotherapy and photodynamic therapy. Some possible surgical options for treatment of skin cancer are mohs micrographic surgery, simple excision, electrodesiccation and curettage, cryosurgery, laser surgery. Radiation therapy can be external beam radiation therapy or brachytherapy. Other types of treatments include biologic therapy or immunotherapy, chemoimmunotherapy, topical chemotherapy with fluorouracil and photodynamic therapy.

Eye Cancer, Retinoblastoma

Retinoblastoma is a malignant tumor of the retina. The tumor can be in one eye only or in both eyes. Treatment options that can be used in combination with the PARP inhibitors of the present invention include enucleation (surgery to remove the eye), radiation therapy, cryotherapy, photocoagulation, immunotherapy, thermotherapy and chemotherapy. Radiation therapy can be external beam radiation therapy or brachytherapy.

Eye Cancer, Intraocular Melanoma

Intraocular melanoma is a disease in which cancer cells are found in the part of the eye called the uvea. The uvea includes the iris, the ciliary body, and the choroid. Intraocular melanoma occurs most often in people who are middle aged. Treatments that can be used in combination with the PARP inhibitors of the present invention include surgery, immunotherapy, radiation therapy and laser therapy. Surgery is the most common treatment of intraocular melanoma. Some possible surgical options are iridectomy, iridotrabeculectomy, iridocyclectomy, choroidectomy, enucleation and orbital exenteration. Radiation therapy can be external beam radiation therapy or brachytherapy. Laser therapy can be an intensely powerful beam of light to destroy the tumor, thermotherapy or photocoagulation.

Endometrium Cancer

Endometrial cancer is a cancer that starts in the endometrium, the inner lining of the uterus. Some of the examples of the cancer of uterus and endometrium include, but are not limited to, adenocarcinomas, adenoacanthomas, adenosquamous carcinomas, papillary serous adenocarcinomas, clear cell adenocarcinomas, uterine sarcomas, stromal sarcomas, malignant mixed mesodermal tumors, and leiomyosarcomas.

Liver Cancer

Primary liver cancer can occur in both adults and children. Different types of treatments that can be used in combination with the PARP inhibitors of the present invention include surgery, immunotherapy, radiation therapy, chemotherapy and percutaneous ethanol injection. The types of surgery that can be used are cryosurgery, partial hepatectomy, total hepatectomy and radiofrequency ablation. Radiation therapy can be external beam radiation therapy, brachytherapy, radiosensitizers or radiolabel antibodies. Other types of treatment include hyperthermia therapy and immunotherapy.

Kidney Cancer

Kidney cancer (also called renal cell cancer or renal adenocarcinoma) is a disease in which malignant cells are found in the lining of tubules in the kidney. Treatments that can be used in combination with the PARP inhibitors of the present invention include surgery, radiation therapy, chemotherapy and immunotherapy. Some possible surgical options to treat kidney cancer are partial nephrectomy, simple nephrectomy and radical nephrectomy. Radiation therapy can be external beam radiation therapy or brachytherapy. Stem cell transplant can be used to treat kidney cancer.

Thyroid Cancer

Thyroid cancer is a disease in which cancer (malignant) cells are found in the tissues of the thyroid gland. The four main types of thyroid cancer are papillary, follicular, medullary and anaplastic. Thyroid cancer can be treated by surgery, immunotherapy, radiation therapy, hormone therapy and chemotherapy. Some possible surgical options that can be used in combination with the PARP inhibitors of the present invention include but are not limited to, lobectomy, near-total thyroidectomy, total thyroidectomy and lymph node dissection. Radiation therapy can be external radiation therapy or can require intake of a liquid that contains radioactive iodine. Hormone therapy uses hormones to stop cancer cells from growing. In treating thyroid cancer, hormones can be used to stop the body from making other hormones that might make cancer cells grow.

AIDS-Related Lymphoma

AIDS-related lymphoma is a disease in which malignant cells form in the lymph system of patients who have acquired immunodeficiency syndrome (AIDS). AIDS is caused by the human immunodeficiency virus (HIV), which attacks and weakens the body's immune system. The immune system is then unable to fight infection and diseases that invade the body. People with HIV disease have an increased risk of developing infections, lymphoma, and other types of cancer. Lymphomas are cancers that affect the white blood cells of the lymph system. Lymphomas are divided into two general types: Hodgkin's lymphoma and non-Hodgkin's lymphoma. Both Hodgkin's lymphoma and non-Hodgkin's lymphoma can occur in AIDS patients, but non-Hodgkin's lymphoma is more common. When a person with AIDS has non-Hodgkin's lymphoma, it is called an AIDS-related lymphoma. Non-Hodgkin's lymphomas can be indolent (slow-growing) or aggressive (fast-growing). AIDS-related lymphoma is usually aggressive. The three main types of AIDS-related lymphoma are diffuse large B-cell lymphoma, B-cell immunoblastic lymphoma and small non-cleaved cell lymphoma.

Highly-active antiretroviral therapy (HAART) is used to slow progression of HIV. Medicine to prevent and treat infections, which can be serious, is also used. AIDS-related lymphomas can be treated by chemotherapy, immunotherapy, radiation therapy and high-dose chemotherapy with stem cell transplant. Radiation therapy can be external beam radiation therapy or brachytherapy. AIDS-related lymphomas can be treated by monoclonal antibody therapy.

Kaposi's Sarcoma

Kaposi's sarcoma is a disease in which cancer cells are found in the tissues under the skin or mucous membranes that line the mouth, nose, and anus. Kaposi's sarcoma can occur in people who are taking immunosuppressants. Kaposi's sarcoma in patients who have Acquired Immunodeficiency Syndrome (AIDS) is called epidemic Kaposi's sarcoma. Kaposi's sarcoma can be treated with surgery, chemotherapy, radiation therapy and immunotherapy. External radiation therapy is a common treatment of Kaposi's sarcoma. Treatments that can be used in combination with the PARP inhibitors of the present invention include but are not limited to, local excision, electrodeiccation and curettage, and cryotherapy.

Viral-Induced Cancers

The virus-malignancy systems include hepatitis B virus (HBV), hepatitis C virus (HCV), and hepatocellular carcinoma; human lymphotropic virus-type 1 (HTLV-1) and adult T-cell leukemia/lymphoma; and human papilloma virus (HPV) and cervical cancer.

Virus-Induced Hepatocellular Carcinoma

HBV and HCV and hepatocellular carcinoma or liver cancer can appear to act via chronic replication in the liver by causing cell death and subsequent regeneration. Treatments that can be used in combination with the PARP inhibitors of the present invention include but are not limited to, include surgery, immunotherapy, radiation therapy, chemotherapy and percutaneous ethanol injection. The types of surgery that can be used are cryosurgery, partial hepatectomy, total hepatectomy and radiofrequency ablation. Radiation therapy can be external beam radiation therapy, brachytherapy, radiosensitizers or radiolabel antibodies. Other types of treatment include hyperthermia therapy and immunotherapy.

Viral-Induced Adult T Cell Leukemia/Lymphoma

Adult T cell leukemia is a cancer of the blood and bone marrow. The treatments for adult T cell leukemia/lymphoma that can be used in combination with the PARP inhibitors of the present invention include but are not limited to, radiation therapy, immunotherapy, and chemotherapy. Radiation therapy can be external beam radiation therapy or brachytherapy. Other methods of treating adult T cell leukemia/lymphoma include immunotherapy and high-dose chemotherapy with stem cell transplantation.

Viral-Induced Cervical Cancer

Infection of the cervix with human papillomavirus (HPV) is a cause of cervical cancer. The treatments for cervical cancers that can be used in combination with the PARP inhibitors of the present invention include but are not limited to, surgery, immunotherapy, radiation therapy and chemotherapy. The types of surgery that can be used are conization, total hysterectomy, bilateral salpingo-oophorectomy, radical hysterectomy, pelvic exenteration, cryosurgery, laser surgery and loop electrosurgical excision procedure. Radiation therapy can be external beam radiation therapy or brachytherapy.

CNS Cancers

Brain and spinal cord tumors are abnormal growths of tissue found inside the skull or the bony spinal column, which are the primary components of the central nervous system (CNS). Benign tumors are non-cancerous, and malignant tumors are cancerous. Tumors that originate in the brain or spinal cord are called primary tumors. Primary tumors can result from specific genetic disease (e.g., neurofibromatosis, tuberous sclerosis) or from exposure to radiation or cancer-causing chemicals.

The primary brain tumor in adults comes from cells in the brain called astrocytes that make up the blood-brain barrier and contribute to the nutrition of the central nervous system. These tumors are called gliomas (astrocytoma, anaplastic astrocytoma, or glioblastoma multiforme). Some of the tumors are, but not limited to, Oligodendroglioma, Ependymoma, Meningioma, Lymphoma, Schwannoma, and Medulloblastoma.

Neuroepithelial Tumors of the CNS

Astrocytic tumors, such as astrocytoma, anaplastic (malignant) astrocytoma, such as hemispheric, diencephalic, optic, brain stem, cerebellar; glioblastoma multiforme; pilocytic astrocytoma, such as hemispheric, diencephalic, optic, brain stem, cerebellar; subependymal giant cell astrocytoma; and pleomorphic xanthoastrocytoma. Oligodendroglial tumors, such as oligodendroglioma; and anaplastic (malignant) oligodendroglioma. Ependymal cell tumors, such as ependymoma; anaplastic ependymoma; myxopapillary ependymoma; and subependymoma. Mixed gliomas, such as mixed oligoastrocytoma; anaplastic (malignant) oligoastrocytoma; and others (e.g. ependymo-astrocytomas). Neuroepithelial tumors of uncertain origin, such as polar spongioblastoma; astroblastoma; and gliomatosis cerebri. Tumors of the choroid plexus, such as choroid plexus papilloma; and choroid plexus carcinoma (anaplastic choroid plexus papilloma). Neuronal and mixed neuronal-glial tumors, such as gangliocytoma; dysplastic gangliocytoma of cerebellum (Lhermitte-Duclos); ganglioglioma; anaplastic (malignant) ganglioglioma; desmoplastic infantile ganglioglioma, such as desmoplastic infantile astrocytoma; central neurocytoma; dysembryoplastic neuroepithelial tumor; olfactory neuroblastoma (esthesioneuroblastoma. Pineal Parenchyma Tumors, such as pineocytoma; pineoblastoma; and mixed pineocytoma/pineoblastoma. Tumors with neuroblastic or glioblastic elements (embryonal tumors), such as medulloepithelioma; primitive neuroectodermal tumors with multipotent differentiation, such as medulloblastoma; cerebral primitive neuroectodermal tumor; neuroblastoma; retinoblastoma; and ependymoblastoma.

Other CNS Neoplasms

Tumors of the sellar region, such as pituitary adenoma; pituitary carcinoma; and craniopharyngioma. Hematopoietic tumors, such as primary malignant lymphomas; plasmacytoma; and granulocytic sarcoma. Germ Cell Tumors, such as germinoma; embryonal carcinoma; yolk sac tumor (endodermal sinus tumor); choriocarcinoma; teratoma; and mixed germ cell tumors. Tumors of the Meninges, such as meningioma; atypical meningioma; and anaplastic (malignant) meningioma. Non-menigothelial tumors of the meninges, such as Benign Mesenchymal; Malignant Mesenchymal; Primary Melanocytic Lesions; Hemopoietic Neoplasms; and Tumors of Uncertain Histogenesis, such as hemangioblastoma (capillary hemangioblastoma). Tumors of Cranial and Spinal Nerves, such as schwannoma (neurinoma, neurilemoma); neurofibroma; malignant peripheral nerve sheath tumor (malignant schwannoma), such as epithelioid, divergent mesenchymal or epithelial differentiation, and melanotic. Local Extensions from Regional Tumors; such as paraganglioma (chemodectoma); chordoma; chodroma; chondrosarcoma; and carcinoma. Metastatic tumors, Unclassified Tumors and Cysts and Tumor-like Lesions, such as Rathke cleft cyst; Epidermoid; dermoid; colloid cyst of the third ventricle; enterogenous cyst; neuroglial cyst; granular cell tumor (choristoma, pituicytoma); hypothalamic neuronal hamartoma; nasal glial herterotopia; and plasma cell granuloma.

Chemotherapeutics available are, but not limited to, alkylating agents such as, Cyclophosphamide, Ifosphamide, Melphalan, Chlorambucil, BCNU, CCNU, Decarbazine, Procarbazine, Busulfan, and Thiotepa; antimetabolites such as, Methotraxate, 5-Fluorouracil, Cytarabine, Gemcitabine (Gemzar®), 6-mercaptopurine, 6-thioguanine, Fludarabine, and Cladribine; anthracyclins such as, daunorubicin. Doxorubicin, Idarubicin, Epirubicin and Mitoxantrone; antibiotics such as, Bleomycin; camptothecins such as, irinotecan and topotecan; taxanes such as, paclitaxel and docetaxel; and platinums such as, Cisplatin, carboplatin, and Oxaliplatin.

PNS Cancers

The peripheral nervous system consists of the nerves that branch out from the brain and spinal cord. These nerves form the communication network between the CNS and the body parts. The peripheral nervous system is further subdivided into the somatic nervous system and the autonomic nervous system. The somatic nervous system consists of nerves that go to the skin and muscles and is involved in conscious activities. The autonomic nervous system consists of nerves that connect the CNS to the visceral organs such as the heart, stomach, and intestines. It mediates unconscious activities.

Acoustic neuromas are benign fibrous growths that arise from the balance nerve, also called the eighth cranial nerve or vestibulocochlear nerve. The malignant peripheral nerve sheath tumor (MPNST) is the malignant counterpart to benign soft tissue tumors such as neurofibromas and schwannomas. It is most common in the deep soft tissue, usually in close proximity of a nerve trunk. The most common sites include the sciatic nerve, brachial plexus, and sarcal plexus.

The MPNST can be classified into three major categories with epithelioid, mesenchymal or glandular characteristics. Some of the MPNST include but not limited to, subcutaneous malignant epithelioid schwannoma with cartilaginous differentiation, glandular malignant schwannoma, malignant peripheral nerve sheath tumor with perineurial differentiation, cutaneous epithelioid malignant nerve sheath tumor with rhabdoid features, superficial epithelioid MPNST, triton Tumor (MPNST with rhabdomyoblastic differentiation), schwannoma with rhabdomyoblastic differentiation. Rare MPNST cases contain multiple sarcomatous tissue types, especially osteosarcoma, chondrosarcoma and angiosarcoma. These have sometimes been indistinguishable from the malignant mesenchymoma of soft tissue.

Other types of PNS cancers include but not limited to, malignant fibrous cytoma, malignant fibrous histiocytoma, malignant meningioma, malignant mesothelioma, and malignant mixed Müllerian tumor.

Oral Cavity and Oropharyngeal Cancer

Cancers of the oral cavity include but are not limited to, hypopharyngeal cancer, laryngeal cancer, nasopharyngeal cancer, and oropharyngeal cancer.

Stomach Cancer

There are three main types of stomach cancers: lymphomas, gastric stromal tumors, and carcinoid tumors. Lymphomas are cancers of the immune system tissue that are sometimes found in the wall of the stomach. Gastric stromal tumors develop from the tissue of the stomach wall. Carcinoid tumors are tumors of hormone-producing cells of the stomach.

Testicular Cancer

Testicular cancer is cancer that typically develops in one or both testicles in young men. Cancers of the testicle develop in certain cells known as germ cells. The two types of germ cell tumors (GCTs) that occur in men are seminomas (60%) and non-seminomas (40%). Tumors can also arise in the supportive and hormone-producing tissues, or stroma, of the testicles. Such tumors are known as gonadal stromal tumors. The two types are Leydig cell tumors and Sertoli cell tumors. Secondary testicular tumors are those that start in another organ and then spread to the testicle. Lymphoma is a secondary testicular cancer.

Thymus Cancer

The thymus is a small organ located in the upper/front portion of your chest, extending from the base of the throat to the front of the heart. The thymus contains two main types of cells, thymic epithelial cells and lymphocytes. Thymic epithelial cells can give origin to thymomas and thymic carcinomas. Lymphocytes, whether in the thymus or in the lymph nodes, can become malignant and develop into cancers called Hodgkin disease and non-Hodgkin lymphomas. The thymus cancer includes Kulchitsky cells, or neuroendocrine cells, which normally release certain hormones. These cells can give rise to cancers, called carcinoids or carcinoid tumors.

Treatments that can be used in combination with the PARP inhibitors of the present invention include but are not limited to, surgery, immunotherapy, chemotherapy, radiation therapy, combination of chemotherapy and radiation therapy or biological therapy. Anticancer drugs that have been used in the treatment of thymomas and thymic carcinomas are doxorubicin (Adriamycin), cisplatin, ifosfamide, and corticosteroids (prednisone).

Examples of Inflammation

Examples of inflammation include, but are not limited to, systemic inflammatory conditions and conditions associated locally with migration and attraction of monocytes, leukocytes and/or neutrophils. Inflammation can result from infection with pathogenic organisms (including gram-positive bacteria, gram-negative bacteria, viruses, fungi, and parasites such as protozoa and helminths), transplant rejection (including rejection of solid organs such as kidney, liver, heart, lung or cornea, as well as rejection of bone marrow transplants including graft-versus-host disease (GVHD)), or from localized chronic or acute autoimmune or allergic reactions. Autoimmune diseases include acute glomerulonephritis; rheumatoid or reactive arthritis; chronic glomerulonephritis; inflammatory bowel diseases such as Crohn's disease, ulcerative colitis and necrotizing enterocolitis; granulocyte transfusion associated syndromes; inflammatory dermatoses such as contact dermatitis, atopic dermatitis, psoriasis; systemic lupus erythematosus (SLE), autoimmune thyroiditis, multiple sclerosis, and some forms of diabetes, or any other autoimmune state where attack by the subjects own immune system results in pathologic tissue destruction. Allergic reactions include allergic asthma, chronic bronchitis, acute and delayed hypersensitivity. Systemic inflammatory disease states include inflammation associated with trauma, burns, reperfusion following ischemic events (e.g. thrombotic events in heart, brain, intestines or peripheral vasculature, including myocardial infarction and stroke), sepsis, ARDS or multiple organ dysfunction syndrome. Inflammatory cell recruitment also occurs in atherosclerotic plaques.

In some preferred embodiments, the inflammation includes Non-Hodgkin's lymphoma, Wegener's granulomatosis, Hashimoto's thyroiditis, hepatocellular carcinoma, thymus atrophy, chronic pancreatitis, rheumatoid arthritis, reactive lymphoid hyperplasia, osteoarthritis, ulcerative colitis, papillary carcinoma, Crohn's disease, ulcerative colitis, acute cholecystitis, chronic cholecystitis, cirrhosis, chronic sialadenitis, peritonitis, acute pancreatitis, chronic pancreatitis, chronic Gastritis, adenomyosis, endometriosis, acute cervicitis, chronic cervicitis, lymphoid hyperplasia, multiple sclerosis, hypertrophy secondary to idiopathic thrombocytopenic purpura, primary IgA nephropathy, systemic lupus erythematosus, psoriasis, pulmonary emphysema, chronic pyelonephritis, and chronic cystitis.

Examples of Endocrine and Neuroendocrine Disorders

Examples of endocrine disorders include disorders of adrenal, breast, gonads, pancreas, parathyroid, pituitary, thyroid, dwarfism etc. The adrenal disorders include, but are not limited to, Addison's disease, hirutism, cancer, multiple endocrine neoplasia, congenital adrenal hyperplasia, and pheochromocytoma. The breast disorders include, but are not limited to, breast cancer, fibrocystic breast disease, and gynecomastia. The gonad disorders include, but are not limited to, congenital adrenal hyperplasia, polycystic ovarian syndrome, and turner syndrome. The pancreas disorders include, but are not limited to, diabetes (type I and type II), hypoglycemia, and insulin resistance. The parathyroid disorders include, but are not limited to, hyperparathyroidism, and hypoparathyroidism. The pituitary disorders include, but are not limited to, acromegaly, Cushing's syndrome, diabetes insipidus, empty sella syndrome, hypopituitarism, and prolactinoma. The thyroid disorders include, but are not limited to, cancer, goiter, hyperthyroid, hypothyroid, nodules, thyroiditis, and Wilson's syndrome. The examples of neuroendocrine disorders include, but are not limited to, depression and anxiety disorders related to a hormonal imbalance, catamenial epilepsy, menopause, menstrual migraine, reproductive endocrine disorders, gastrointestinal disorders such as, gut endocrine tumors including carcinoid, gastrinoma, and somatostatinoma, achalasia, and Hirschsprung's disease. In some embodiments, the endocrine and neuroendocrine disorders include nodular hyperplasia, Hashimoto's thyroiditis, islet cell tumor, and papillary carcinoma.

The endocrine and neuroendocrine disorders in children include endocrinologic conditions of growth disorder and diabetes insipidus. Growth delay can be observed with congenital ectopic location or aplasia/hypoplasia of the pituitary gland, as in holoprosencephaly, septo-optic dysplasia and basal encephalocele. Acquired conditions, such as craniopharyngioma, optic/hypothalamic glioma can be present with clinical short stature and diencephalic syndrome. Precocious puberty and growth excess can be seen in the following conditions: arachnoid cyst, hydrocephalus, hypothalamic hamartoma and germinoma. Hypersecretion of growth hormone and adrenocorticotropic hormone by a pituitary adenoma can result in pathologically tall stature and truncal obesity in children. Diabetes insipidus can occur secondary to infiltrative processes such as langerhans cell of histiocytosis, tuberculosis, germinoma, post traumatic/surgical injury of the pituitary stalk and hypoxic ischemic encephalopathy.

Examples of Nutritional and Metabolic Disorders

The examples of nutritional and metabolic disorders include, but are not limited to, aspartylglusomarinuria, biotinidase deficiency, carbohydrate deficient glycoprotein syndrome (CDGS), Crigler-Najjar syndrome, cystinosis, diabetes insipidus, fabry, fatty acid metabolism disorders, galactosemia, gaucher, glucose-6-phosphate dehydrogenase (G6PD), glutaric aciduria, hurler, hurler-scheie, hunter, hypophosphatemia, 1-cell, krabbe, lactic acidosis, long chain 3 hydroxyacyl CoA dehydrogenase deficiency (LCHAD), lysosomal storage diseases, mannosidosis, maple syrup urine, maroteaux-lamy, metachromatic leukodystrophy, mitochondrial, morquio, mucopolysaccharidosis, neuro-metabolic, niemann-pick, organic acidemias, purine, phenylketonuria (PKU), pompe, pseudo-hurler, pyruvate dehydrogenase deficiency, sandhoff, sanfilippo, scheie, sly, tay-sachs, trimethylaminuria (fish-malodor syndrome), urea cycle conditions, vitamin D deficiency rickets, metabolic disease of muscle, inherited metabolic disorders, acid-base imbalance, acidosis, alkalosis, alkaptonuria, alpha-mannosidosis, amyloidosis, anemia, iron-deficiency, ascorbic acid deficiency, avitaminosis, beriberi, biotinidase deficiency, deficient glycoprotein syndrome, carnitine disorders, cystinosis, cystinuria, fabry disease, fatty acid oxidation disorders, fucosidosis, galactosemias, gaucher disease, gilbert disease, glucosephosphate dehydrogenase deficiency, glutaric academia, glycogen storage disease, hartnup disease, hemochromatosis, hemosiderosis, hepatolenticular degeneration, histidinemia, homocystinuria, hyperbilirubinemia, hypercalcemia, hyperinsulinism, hyperkalemia, hyperlipidemia, hyperoxaluria, hypervitaminosis A, hypocalcemia, hypoglycemia, hypokalemia, hyponatremia, hypophosphotasia, insulin resistance, iodine deficiency, iron overload, jaundice, chronic idiopathic, leigh disease, Lesch-Nyhan syndrome, leucine metabolism disorders, lysosomal storage diseases, magnesium deficiency, maple syrup urine disease, MELAS syndrome, menkes kinky hair syndrome, metabolic syndrome X, mucolipidosis, mucopolysacchabridosis, Niemann-Pick disease, obesity, ornithine carbamoyltransferase deficiency disease, osteomalacia, pellagra, peroxisomal disorders, porphyria, erythropoietic, porphyries, progeria, pseudo-gaucher disease, refsum disease, reye syndrome, rickets, sandhoff disease, tangier disease, Tay-sachs disease, tetrahydrobiopterin deficiency, trimethylaminuria (fish odor syndrome), tyrosinemias, urea cycle disorders, water-electrolyte imbalance, wernicke encephalopathy, vitamin A deficiency, vitamin B12 deficiency, vitamin B deficiency, wolman disease, and zellweger syndrome.

In some preferred embodiments, the metabolic diseases include diabetes and obesity.

Examples of Hematolymphoid System

A hematolymphoid system includes hemic and lymphatic diseases. A “hematological disorder” includes a disease, disorder, or condition which affects a hematopoietic cell or tissue. Hematological disorders include diseases, disorders, or conditions associated with aberrant hematological content or function. Examples of hematological disorders include disorders resulting from bone marrow irradiation or chemotherapy treatments for cancer, disorders such as pernicious anemia, hemorrhagic anemia, hemolytic anemia, aplastic anemia, sickle cell anemia, sideroblastic anemia, anemia associated with chronic infections such as malaria, trypanosomiasis, HIV, hepatitis virus or other viruses, myelophthisic anemias caused by marrow deficiencies, renal failure resulting from anemia, anemia, polycethemia, infectious mononucleosis (IM), acute non-lymphocytic leukemia (ANLL), acute Myeloid Leukemia (AML), acute promyelocytic leukemia (APL), acute myelomonocytic leukemia (AMMoL), polycethemia vera, lymphoma, acute lymphocytic leukemia (ALL), chronic lymphocytic leukemia, Wilm's tumor, Ewing's sarcoma, retinoblastoma, hemophilia, disorders associated with an increased risk of thrombosis, herpes, thalessemia, antibody-mediated disorders such as transfusion reactions and erythroblastosis, mechanical trauma to red blood cells such as micro-angiopathic hemolytic anemias, thrombotic thrombocytopenic purpura and disseminated intravascular coagulation, infections by parasites such as plasmodium, chemical injuries from, e.g., lead poisoning, and hypersplenism.

Lymphatic diseases include, but are not limited to, lymphadenitis, lymphagiectasis, lymphangitis, lymphedema, lymphocele, lymphoproliferative disorders, mucocutaneous lymph node syndrome, reticuloendotheliosis, splenic diseases, thymus hyperplasia, thymus neoplasms, tuberculosis, lymph node, pseudolymphoma, and lymphatic abnormalities.

In some preferred embodiments, the disorders of hematolymphoid system include, non-Hodgkin's lymphoma, chronic lymphocytic leukemia, and reactive lymphoid hyperplasia.

Examples of CNS Diseases

The examples of CNS diseases include, but are not limited to, neurodegenerative diseases, drug abuse such as, cocaine abuse, multiple sclerosis, schizophrenia, acute disseminated encephalomyelitis, transverse myelitis, demyelinating genetic diseases, spinal cord injury, virus-induced demyelination, progressive multifocal leucoencephalopathy, human lymphotrophic T-cell virus I (HTLVI)-associated myelopathy, and nutritional metabolic disorders.

In some preferred embodiments, the CNS diseases include Parkinson disease, Alzheimer's disease, cocaine abuse, and schizophrenia.

Examples of Neurodegenerative Diseases

Neurodegenerative diseases in the methods of the present invention include, but are not limited to, Alzheimer's disease, Pick's disease, diffuse lewy body disease, progressive supranuclear palsy (Steel-Richardson syndrome), multisystem degeneration (Shy-Drager syndrome), motor neuron diseases including amyotrophic lateral sclerosis, degenerative ataxias, cortical basal degeneration, ALS-Parkinson's-dementia complex of guam, subacute sclerosing panencephalitis, Huntington's disease, Parkinson's disease, synucleinopathies, primary progressive aphasia, striatonigral degeneration, Machado-Joseph disease/spinocerebellar ataxia type 3 and olivopontocerebellar degenerations, Gilles De La Tourette's disease, bulbar and pseudobulbar palsy, spinal and spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, and prion diseases (including Creutzfeldt-Jakob, Gerstmann-Straussler-Scheinker disease, kuru and fatal familial insomnia), Alexander disease, alper's disease, amyotrophic lateral sclerosis, ataxia telangiectasia, batten disease, canavan disease, cockayne syndrome, corticobasal degeneration, Creutzfeldt-Jakob disease, Huntington disease, Kennedy's disease, Krabbe disease, lewy body dementia, Machado-Joseph disease, spinocerebellar ataxia type 3, multiple sclerosis, multiple system atrophy, Parkinson disease, Pelizaeus-Merzbacher Disease, Refsum's disease, Schilder's disease, Spielmeyer-Vogt-Sjogren-Batten disease, Steele-Richardson-Olszewski disease, and tabes dorsalis.

Examples of Disorders of Urinary Tract

Disorders of urinary tract in the methods of the present invention include, but are not limited to, disorders of kidney, ureters, bladder, and urethera. For example, urethritis, cystitis, pyelonephritis, renal agenesis, hydronephrosis, polycystic kidney disease, multicystic kidneys, low urinary tract obstruction, bladder exstrophy and epispadias, hypospadias, bacteriuria, prostatitis, intrarenal and peripheral abscess, benign prostate hypertrophy, renal cell carcinoma, transitional cell carcinoma, Wilm's tumor, uremia, and glomerolonephritis.

Examples of Respiratory Diseases

The respiratory diseases and conditions include, but are not limited to, asthma, chronic obstructive pulmonary disease (COPD), adenocarcinoma, adenosquamous carcinoma, squamous cell carcinoma, large cell carcinoma, cystic fibrosis (CF), dispnea, emphysema, wheezing, pulmonary hypertension, pulmonary fibrosis, hyper-responsive airways, increased adenosine or adenosine receptor levels, pulmonary bronchoconstriction, lung inflammation and allergies, and surfactant depletion, chronic bronchitis, bronchoconstriction, difficult breathing, impeded and obstructed lung airways, adenosine test for cardiac function, pulmonary vasoconstriction, impeded respiration, acute respiratory distress syndrome (ARDS), administration of certain drugs, such as adenosine and adenosine level increasing drugs, and other drugs for, e.g. treating supraventricular tachycardia (SVT), and the administration of adenosine stress tests, infantile respiratory distress syndrome (infantile RDS), pain, allergic rhinitis, decreased lung surfactant, decreased ubiquinone levels, or chronic bronchitis, among others.

Examples of Disorders of Female Genital System

The disorders of the female genital system include diseases of the vulva, vagina, cervix uteri, corpus uteri, fallopian tube, and ovary. Some of the examples include, adnexal diseases such as, fallopian tube disease, ovarian disease, leiomyoma, mucinous cystadenocarcinoma, serous cystadenocarcinoma, parovarian cyst, and pelvic inflammatory disease; endometriosis; genital neoplasms such as, fallopian tube neoplasms, uterine neoplasms, vaginal neoplasms, vulvar neoplasms, and ovarian neoplasms; gynatresia; genital herpes; infertility; sexual dysfunction such as, dyspareunia, and impotence; tuberculosis; uterine diseases such as, cervix disease, endometrial hyperplasia, endometritis, hematometra, uterine hemorrhage, uterine neoplasms, uterine prolapse, uterine rupture, and uterine inversion; vaginal diseases such as, dyspareunia, hematocolpos, vaginal fistula, vaginal neoplasms, vaginitis, vaginal discharge, and candidiasis or vulvovaginal; vulvar diseases such as, kraurosis vulvae, pruritus, vulvar neoplasm, vulvitis, and candidiasis; and urogenital diseases such as urogenital abnormalities and urogenital neoplasms.

Examples of Disorders of Male Genital System

The disorders of the male genital system include, but are not limited to, epididymitis; genital neoplasms such as, penile neoplasms, prostatic neoplasms, and testicular neoplasms; hematocele; genital herpes; hydrocele; infertility; penile diseases such as, balanitis, hypospadias, peyronie disease, penile neoplasms, phimosis, and priapism; prostatic diseases such as, prostatic hyperplasia, prostatic neoplasms, and prostatitis; organic sexual dysfunction such as, dyspareunia, and impotence; spermatic cord torsion; spermatocele; testicular diseases such as, cryptorchidism, orchitis, and testicular neoplasms; tuberculosis; varicocele; urogenital diseases such as, urogenital abnormalities, and urogenital neoplasms; and fournier gangrene.

Examples of Cardiovascular Disorders (CVS)

The cardiovascular disorders include those disorders that can either cause ischemia or are caused by reperfusion of the heart. Examples include, but are not limited to, atherosclerosis, coronary artery disease, granulomatous myocarditis, chronic myocarditis (non-granulomatous), primary hypertrophic cardiomyopathy, peripheral artery disease (PAD), stroke, angina pectoris, myocardial infarction, cardiovascular tissue damage caused by cardiac arrest, cardiovascular tissue damage caused by cardiac bypass, cardiogenic shock, and related conditions that would be known by those of ordinary skill in the art or which involve dysfunction of or tissue damage to the heart or vasculature, especially, but not limited to, tissue damage related to PARP activation.

In some preferred embodiments of the present invention, CVS diseases include, atherosclerosis, granulomatous myocarditis, myocardial infarction, myocardial fibrosis secondary to valvular heart disease, myocardial fibrosis without infarction, primary hypertrophic cardiomyopathy, and chronic myocarditis (non-granulomatous).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A computer-assisted method of a designing of a PARP inhibitor comprising:
a) determining an interaction between a candidate PARP protein and a known PARP inhibitor by evaluating a binding of said candidate PARP protein to said known PARP inhibitor;
b) based on said interaction, designing a candidate PARP inhibitor;
c) determining an interaction between said PARP protein and said candidate PARP inhibitor by evaluating a binding of said PARP protein to said candidate PARP inhibitor; and
d) concluding that said candidate PARP inhibitor inhibits said PARP protein wherein said conclusion is based on said interaction of step c).

2. The method of claim 1, wherein said PARP protein is a three-dimensional structure derived from a crystal of said PARP protein and wherein said three dimensional structure comprises a binding domain of said PARP protein.

3. The method of claim 2, wherein said binding domain of said PARP protein is selected from the group consisting of DNA binding domain, automodification domain, and catalytic domain.

4. The method of claim 3, wherein said binding domain of said PARP protein is a catalytic domain.

5. The method of claim 1, wherein said known PARP inhibitor is a three-dimensional structure.

6. The method of claim 1, wherein said PARP protein is a PARP 1 protein.

7. The method of claim 1, wherein said designing is performed in conjunction with a computer modeling.

8. The method of claim 1, wherein said designing involves replacing a substituent on said known PARP inhibitor with a other substituent wherein said other substituent improves said binding of said candidate PARP inhibitor with said PARP protein.

9. The method of claim 1, wherein said interaction is steric interaction, van der Waals interaction, electrostatic interaction, solvation interaction, charge interaction, covalent bonding interaction, non-covalent bonding interaction, entropically favorable interaction, enthalpically favorable interaction, or a combination thereof.

10. The method of claim 1, wherein said candidate PARP inhibitor is an analog of said known PARP inhibitor.

11. The method of claim 10, wherein said candidate PARP inhibitor contains a hydrophillic group.

12. The method of claim 11, wherein said hydrophillic group contains at least one nitrogen.

13. The method of claim 1, wherein said known PARP inhibitor is an iodonitrocoumarin.

14. The method of claim 13, wherein said candidate PARP inhibitor is an analog of said iodonitrocoumarin.

15. The method of claim 1, further comprising a step of chemically synthesizing said candidate PARP inhibitor.

16. The method of claim 15, further comprising evaluating a PARP inhibiting activity of said candidate PARP inhibitor and selecting said candidate PARP inhibitor based on said evaluation.

17. The method of claim 16, wherein said evaluating said PARP inhibiting activity involves an assay technique.

18. The method of claim 1, wherein the candidate PARP inhibitor is a compound of formula I, its pharmaceutically acceptable salts or prodrugs thereof: 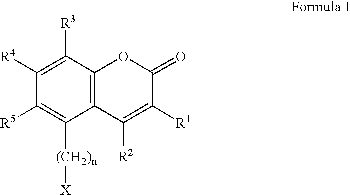
wherein n=0-10; R¹, R², R³, R⁴, R⁵ and X are independently selected from the group consisting of hydrogen, hydroxy, optionally substituted amine, carboxyl, ester, nitroso, nitro, halogen, optionally substituted (C₁-C₆) alkyl, optionally substituted (C₁-C₆) alkoxy, optionally substituted (C₃-C₇) cycloalkyl, optionally substituted (C₃-C₇) heterocyclic, and optionally substituted aryl; and wherein at least two of the R¹, R², R³, R⁴, and R⁵ substituents are always hydrogen.

19. The method of claim 17, wherein the candidate PARP inhibitor is a compound of formula II or its pharmaceutically acceptable salts or prodrugs: 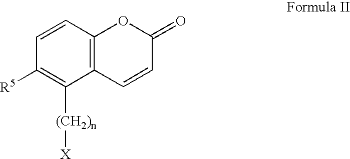
wherein R⁵ is selected from the group consisting of carboxyl, nitroso, and nitro; and X is selected from the group consisting of optionally substituted (C₁-C₇) alkyl, optionally substituted (C₁-C₆) alkoxy, optionally substituted (C₃-C₇) cycloalkyl, optionally substituted (C₃-C₇) heterocyclic, and optionally substituted aryl.

20. The method of claim 18, wherein the candidate PARP inhibitor is a compound of formula III or its pharmaceutically acceptable salts or prodrugs: 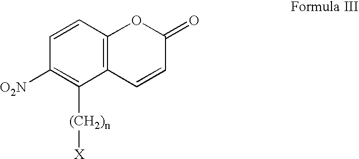
wherein n=0-10, and wherein X is selected from the group consisting of optionally substituted (C₃-C₇) cycloalkyl, optionally substituted (C₃-C₇) heterocyclic, and optionally substituted aryl.

21. The method of claim 19, wherein the optionally substituted aryl is substituted with an optionally substituted alkyl.

22. The method of claim 20, wherein the optionally substituted alkyl is substituted with a substituent selected from the group consisting of alkylamine, pyrrole, dihydropyrrole, or pyrrolidene.

23. The method of claim 21, wherein the candidate PARP inhibitor is a compound of formula IIIa or its pharmaceutically acceptable salts or prodrugs:

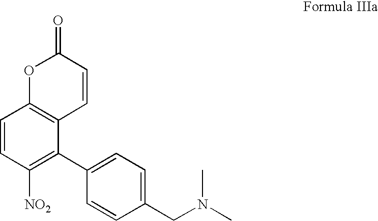

24. The method of claim 21, wherein the candidate PARP inhibitor is a compound of formula IIIb or its pharmaceutically acceptable salts or prodrugs:

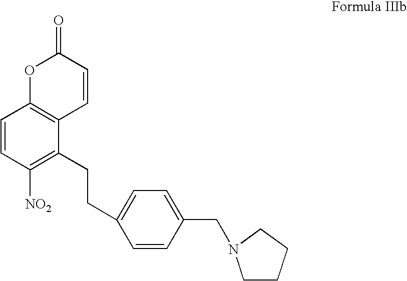

25. The method of claim 19, wherein the optionally substituted (C3-C7) heterocyclic is a five membered heterocyclic ring or a six membered heterocyclic ring.

26. The method of claim 24, wherein the optionally substituted (C3-C7) heterocyclic contains at least one nitrogen.

27. The method of claim 24, wherein the optionally substituted (C3-C7) heterocyclic is selected from the group consisting of azeridine, azetidine, pyrrole, dihydropyrrole, pyrrolidene, pyrazole, pyrazoline, pyrazolidine, imidazole, benzimidazole, triazole, tetrazole, oxazole, isoxazole, benzoxazole, oxadiazole, oxazoline, oxazolidine, thiazole, isothiazole, pyridine, dihydropyridine, tetrahydropyridine, quinazoline, pyrazine, pyrimidine, pyridazine, quinoline, isoquinoline, triazine, tetrazine, and piperazine.

28. The method of claim 26, wherein the optionally substituted (C3-C7) heterocyclic is substituted with a substituent selected from the group consisting of optionally substituted (C1-C6) alkyl, optionally substituted (C1-C6) alkoxy, optionally substituted (C3-C7) cycloalkyl, optionally substituted (C3-C7) heterocyclic, and optionally substituted aryl.

29. The method of claim 27, wherein the candidate PARP inhibitor is a compound of formula IIIc or its pharmaceutically acceptable salts or prodrugs:

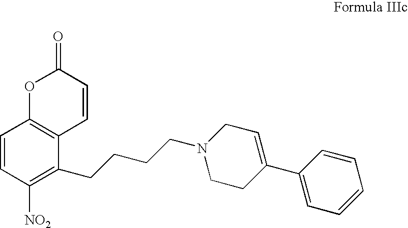

30. The method of claim 27, wherein the candidate PARP inhibitor is a compound of formula IIId or its pharmaceutically acceptable salts or prodrugs:

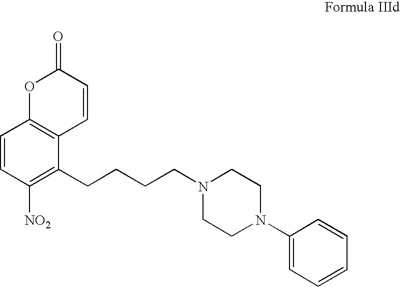

31. The method of claim 27, wherein the candidate PARP inhibitor is a compound of formula IIIe or its pharmaceutically acceptable salts or prodrugs:

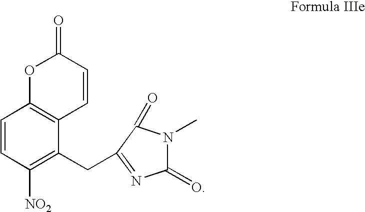

32. The method of claim 27, wherein the candidate PARP inhibitor is a compound of formula IIIf or its pharmaceutically acceptable salts or prodrugs:

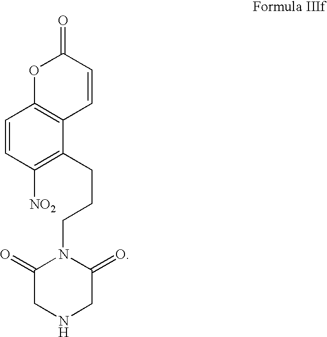

33. A computer system containing a set of information to perform a design of a PARP inhibitor having a user interface comprising a display unit, the set of information comprising:
a) logic for inputting an information regarding a binding of a PARP protein to a known PARP inhibitor;
b) logic for designing a candidate PARP inhibitor based on the binding of the PARP protein and known PARP inhibitor;
c) logic for determining an information regarding a binding of the PARP protein to the candidate PARP inhibitor; and
d) logic for making a conclusion regarding the PARP inhibitory properties of the candidate PARP inhibitor based on the determination of step c).

34. A computer-readable storage medium containing a set of information for a general purpose computer having a user interface comprising a display unit, the set of information comprising:
a) logic for inputting an information regarding a binding of a PARP protein to a known PARP inhibitor;
b) logic for designing a candidate PARP inhibitor based on the binding of the PARP protein and known PARP inhibitor;
c) logic for determining an information regarding a binding of the PARP protein to the candidate PARP inhibitor; and
d) logic for making a conclusion regarding the PARP inhibitory properties of the candidate PARP inhibitor based on the determination of step c).

35. An electronic signal or carrier wave that is propagated over the internet between computers comprising a set of information for a general purpose computer having a user interface comprising a display unit, the set of information comprising a computer-readable storage medium containing a set of information for a general purpose computer having a user interface comprising a display unit, the set of information comprising:
a) logic for inputting an information regarding a binding of a PARP protein to a known PARP inhibitor;
b) logic for designing a candidate PARP inhibitor based on the binding of the PARP protein and known PARP inhibitor;
c) logic for determining an information regarding a binding of the PARP protein to the candidate PARP inhibitor; and
d) logic for making a conclusion regarding the PARP inhibitory properties of the candidate PARP inhibitor based on the determination of step c).

36. A method of treating a disease comprising administering to a patient in need thereof an effective amount of at least one compound of formula I, its pharmaceutically acceptable salts or prodrugs thereof: 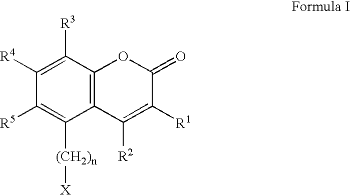
wherein n=0-10; R¹, R², R³, R⁴, R⁵ and X are independently selected from the group consisting of hydrogen, hydroxy, optionally substituted amine, carboxyl, ester, nitroso, nitro, halogen, optionally substituted (C₁-C₆) alkyl, optionally substituted (C₁-C₆) alkoxy, optionally substituted (C₃-C₇) cycloalkyl, optionally substituted (C₃-C₇) heterocyclic, and optionally substituted aryl; and wherein at least two of the R¹, R², R³, R⁴, and R⁵ substituents are always hydrogen.

37. The method of claim 36, wherein the compound is of formula II or its pharmaceutically acceptable salts or prodrugs: 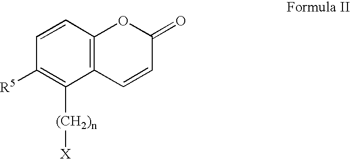
wherein R⁵ is selected from the group consisting of carboxyl, nitroso, and nitro; and X is selected from the group consisting of optionally substituted (C₁-C₇) alkyl, optionally substituted (C₁-C₆) alkoxy, optionally substituted (C₃-C₇) cycloalkyl, optionally substituted (C₃-C₇) heterocyclic, and optionally substituted aryl.

38. The method of claim 37, wherein the optionally substituted alkyl is substituted with a substituent selected from the group consisting of alkylamine, pyrrole, dihydropyrrole, or pyrrolidene.

39. The method of claim 38, wherein the compound is of formula IIIa or its pharmaceutically acceptable salts or prodrugs:

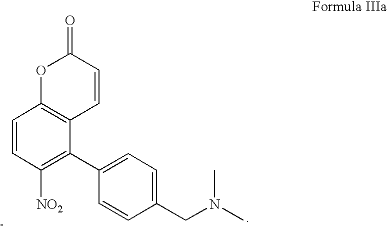

40. The method of claim 38, wherein the compound is of formula IIIb or its pharmaceutically acceptable salts or prodrugs:

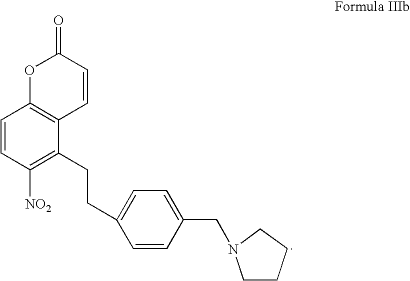

41. The method of claim 36, wherein the optionally substituted (C₃-C₇) heterocyclic is a five membered heterocyclic ring or a six membered heterocyclic ring.

42. The method of claim 41, wherein the optionally substituted (C₃-C₇) heterocyclic contains at least one nitrogen.

43. The method of claim 36, wherein the optionally substituted (C₃-C₇) heterocyclic is selected from the group consisting of azeridine, azetidine, pyrrole, dihydropyrrole, pyrrolidene, pyrazole, pyrazoline, pyrazolidine, imidazole, benzimidazole, triazole, tetrazole, oxazole, isoxazole, benzoxazole, oxadiazole, oxazoline, oxazolidine, thiazole, isothiazole, pyridine, dihydropyridine, tetrahydropyridine, quinazoline, pyrazine, pyrimidine, pyridazine, quinoline, isoquinoline, triazine, tetrazine, and piperazine.

44. The method of claim 43, wherein the optionally substituted (C₃-C₇) heterocyclic is substituted with a substituent selected from the group consisting of optionally substituted (C₁-C₆) alkyl, optionally substituted (C₁-C₆) alkoxy, optionally substituted (C₃-C₇) cycloalkyl, optionally substituted (C₃-C₇) heterocyclic, and optionally substituted aryl.

45. The method of claim 36, wherein the compound is of formula IIIc or its pharmaceutically acceptable salts or prodrugs:

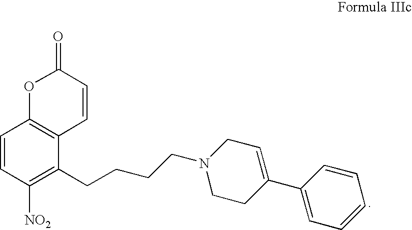

46. The method of claim 36, wherein the compound is of formula IIId or its pharmaceutically acceptable salts or prodrugs:

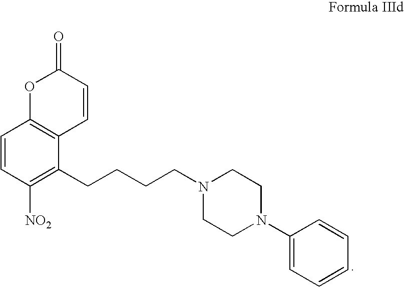

47. The method of claim 36, wherein the compound is of formula IIIe or its pharmaceutically acceptable salts or prodrugs:

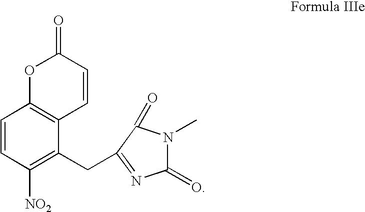

48. The method of claim 36, wherein the compound is of formula IIIf or its pharmaceutically acceptable salts or prodrugs:

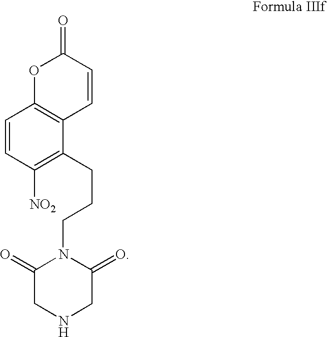

49. The method of claim 36, wherein the treating comprises inhibiting a PARP protein.

50. The method of claim 36, wherein the disease is selected from the group consisting of cancer, inflammation, metabolic disease, CVS disease, CNS disease, disorder of hematolymphoid system, disorder of endocrine and neuroendocrine, disorder of urinary tract, disorder of respiratory system, disorder of female genital system, and disorder of male genital system.

51. A compound of formula I, its pharmaceutically acceptable salts or prodrugs thereof: 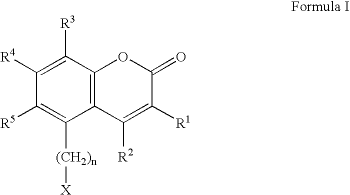
wherein n=0-10; R¹, R², R³, R⁴, R⁵ and X are independently selected from the group consisting of hydrogen, hydroxy, optionally substituted amine, carboxyl, ester, nitroso, nitro, halogen, optionally substituted (C₁-C₆) alkyl, optionally substituted (C₁-C₆) alkoxy, optionally substituted (C₃-C₇) cycloalkyl, optionally substituted (C₃-C₇) heterocyclic, and optionally substituted aryl; and wherein at least two of the R¹, R², R³, R⁴, and R⁵ substituents are always hydrogen.

52. The compound of claim 51, wherein the compound is of formula II or its pharmaceutically acceptable salts or prodrugs: 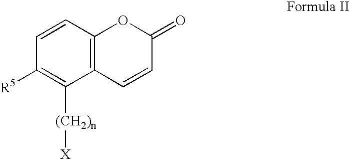
wherein R⁵ is selected from the group consisting of carboxyl, nitroso, and nitro; and X is selected from the group consisting of optionally substituted (C₁-C₇) alkyl, optionally substituted (C₁-C₆) alkoxy, optionally substituted (C₃-C₇) cycloalkyl, optionally substituted (C₃-C₇) heterocyclic, and optionally substituted aryl.

53. The compound of claim 52, wherein the compound is of formula III or its pharmaceutically acceptable salts or prodrugs: 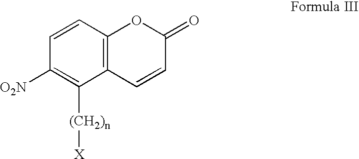
wherein n=0-10, and wherein X is selected from the group consisting of optionally substituted (C₃-C₇) cycloalkyl, optionally substituted (C₃-C₇) heterocyclic, and optionally substituted aryl.

54. The compound of claim 53, wherein the optionally substituted aryl is substituted with an optionally substituted alkyl.

55. The compound of claim 54, wherein the optionally substituted alkyl is substituted with a substituent selected from the group consisting of alkylamine, pyrrole, dihydropyrrole, or pyrrolidene.

56. The compound of claim 55, wherein the compound is of formula IIIa or its pharmaceutically acceptable salts or prodrugs:

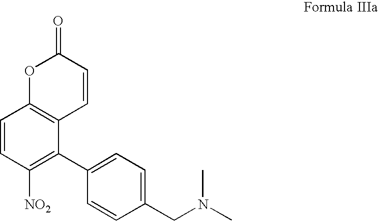

57. The compound of claim 55, wherein the compound is of formula IIIb or its pharmaceutically acceptable salts or prodrugs:

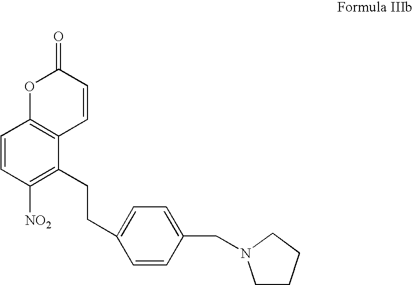

58. The compound of claim 53, wherein the optionally substituted (C₃-C₇) heterocyclic is a five membered heterocyclic ring or a six membered heterocyclic ring.

59. The compound of claim 53, wherein the optionally substituted (C₃-C₇) heterocyclic contains at least one nitrogen.

60. The compound of claim 53, wherein the optionally substituted (C₃-C₇) heterocyclic is selected from the group consisting of azeridine, azetidine, pyrrole, dihydropyrrole, pyrrolidene, pyrazole, pyrazoline, pyrazolidine, imidazole, benzimidazole, triazole, tetrazole, oxazole, isoxazole, benzoxazole, oxadiazole, oxazoline, oxazolidine, thiazole, isothiazole, pyridine, dihydropyridine, tetrahydropyridine, quinazoline, pyrazine, pyrimidine, pyridazine, quinoline, isoquinoline, triazine, tetrazine, and piperazine.

61. The compound of claim 60, wherein the optionally substituted (C₃-C₇) heterocyclic is substituted with a substituent selected from the group consisting of optionally substituted (C₁-C₆) alkyl, optionally substituted (C₁-C₆) alkoxy, optionally substituted (C₃-C₇) cycloalkyl, optionally substituted (C₃-C₇) heterocyclic, and optionally substituted aryl.

62. The compound of claim 53, wherein the compound is of formula IIIc or its pharmaceutically acceptable salts or prodrugs:

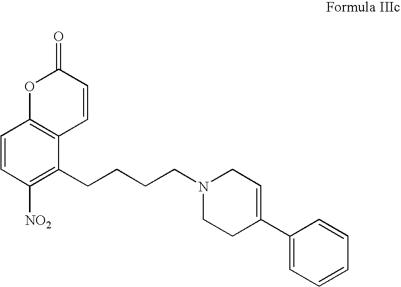

63. The compound of claim 53, wherein the compound is of formula IIId or its pharmaceutically acceptable salts or prodrugs:

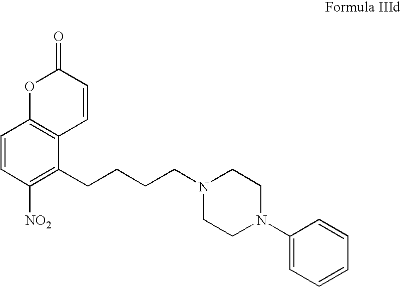

64. The compound of claim 53, wherein the compound is of formula IIIe or its pharmaceutically acceptable salts or prodrugs:

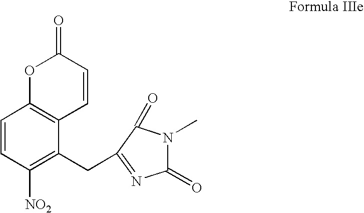

65. The compound of claim 53, wherein the compound is of formula IIIf or its pharmaceutically acceptable salts or prodrugs:

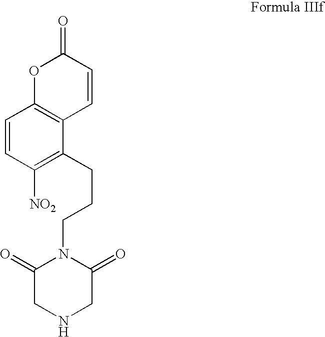

66. A compound comprising at least one structure selected from formula IIIa-f, its pharmaceutically acceptable salts or prodrugs thereof:

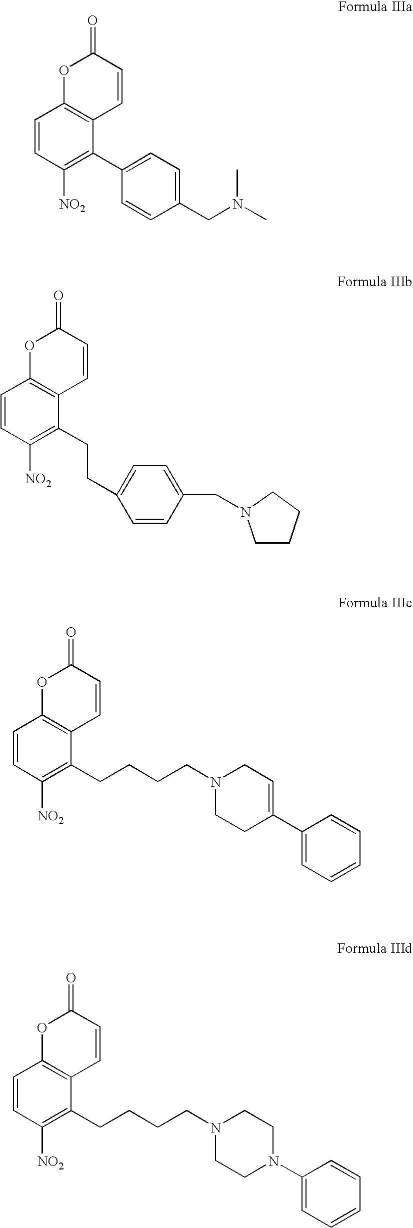

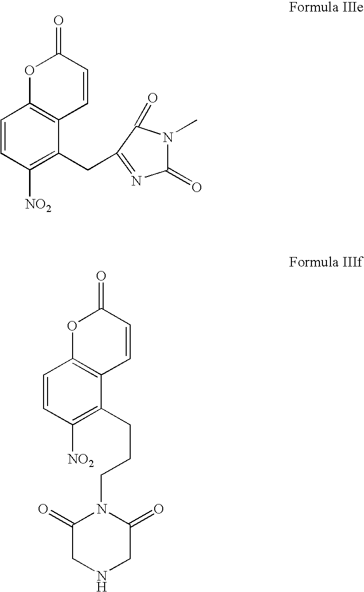

67. A pharmaceutical composition comprising an effective amount of at least one compound or its pharmaceutically acceptable salts or prodrugs of claim 51 and a pharmaceutically acceptable carrier.
